(19) United States
(12) Patent Application Publication
(10) Pub. No.: US 2026/0009812 A1
(43) Pub. Date: Jan. 8, 2026

(54) SYSTEM AND METHOD FOR DRUM POSITION DETECTION

(71) Applicant: BECTON, DICKINSON AND COMPANY, Franklin Lakes, NJ (US)

(72) Inventors: Robert Edward Armstrong, Hunt Valley, MD (US); Alexander W. Clark, Baltimore, MD (US)

(73) Assignee: BECTON, DICKINSON AND COMPANY, Franklin Lakes, NJ (US)

(21) Appl. No.: 18/993,689

(22) PCT Filed: Jul. 18, 2023
(86) PCT No.: PCT/US2023/027996

§ 371 Date: Jan. 13, 2025

Related U.S. Application Data
(60) Provisional application No. 63/390,506, filed on Jul. 19, 2022.

(51) Int. Cl. G01N 35/04

(52) U.S. Cl. CPC G01N 35/04; G01N 2035/0441; G01N 2035/0491

(57) ABSTRACT

A system (6000) and method (7000, 7050) for detecting a position of a rotating drum (6050) of a blood culture apparatus relative to a stationary measurement board (6060) disposed adjacent to the rotating drum (6050). The system (6000) and method (7000, 7050) can adjust at least one stored signal of a sensor (6062) of the measurement board (6060) based on the detected position of the rotating drum (6050).

### CROSS REFERENCE TO RELATED APPLICATIONS

This application is related to PCT Application No. PCT/US2020/045065, which was filed Aug. 5, 2020 and which is incorporated by reference herein. This application claims the priority of and benefit from U.S. Provisional Patent Application No. 63/390,506, filed Jul. 19, 2022, which is incorporated by reference herein.

### TECHNICAL FIELD

The present technology relates to a system and method for detecting the position of a rotating drum (and changes thereto) of a blood culture apparatus relative to a stationary measurement board disposed adjacent to the rotating drum. The systems and methods of the present technology adjust at least one signal of a sensor of the measurement board based on the detected position of the rotating drum.

### BACKGROUND OF THE INVENTION

The presence of biologically active agents such as bacteria in a patient's body fluid, especially blood, is generally determined using blood culture bottles. A small quantity of blood is injected through an enclosing rubber septum into a sterile bottle containing a culture medium, and the bottle is then incubated at about 35° C. and monitored for microorganism growth.

Since it is of utmost importance to learn if a patient has a bacterial infection, hospitals and laboratories have automated apparatus that can process many blood culture bottles simultaneously. One example of such an apparatus is the BD BACTEC™ system, which is manufactured and sold by Becton, Dickinson and Co. U.S. Pat. No. 5,817,508 to Berndt et al. describes a prior art blood culture apparatus, and is incorporated by reference herein. Additional descriptions of Blood Culture Apparatus are provided in U.S. Pat. No. 5,516,692 (“Compact Blood Culture Apparatus”) and U.S. Pat. No. 5,498,543 (“Sub-Compact Blood Culture Apparatus”) both of which are incorporated by reference herein.

Referring to FIG. 1, a culture medium and blood specimen mixture 22 are introduced into sealable glass bottles 1 that include optical chemical sensing means 20 on their inner bottom surface 21. Optical chemical sensing means 20 emanates differing quantities of light depending upon the amount of a gas in bottle 1. For example, the gas being detected by optical sensing means 20 can be carbon dioxide, oxygen or any gas that increases or decreases depending upon the presence or absence of microorganism growth in bottle 1.

As illustrated in FIGS. 1 and 2, a plurality of such bottles 1 are arranged radially on a rotating bell-shaped drum 2 within an incubator 5 in such a way that the bottoms of bottles 1 are oriented towards a drum axis 28. Bell-shaped drum 2 is hollow and is supported by a shaft 24 rotatably supported on one end by two large ball-bearings 3 and 4 mounted to a first side 51 of an instrument mainframe 50. In order to read information coming from each optical chemical sensing means 20 within bottles 1, a linear array of sensor stations 12 is mounted within rotating bell-shaped drum 2 to a second side 52 of instrument mainframe 50 at such a distance inside bell-shaped drum 2 that, during rotation of drum 2, individual bottles 1 are passing by respective sensor stations 15 in array 12. Each sensor station 15 of the linear array of sensor stations 12 comprises an excitation light source 11 and a collection end of an optical fiber 14.

Axis 28 of the bell-shaped drum 2 is oriented horizontally and parallel to a door 13, shown in FIG. 2, located on a front face of incubator 5. Horizontal orientation of axis 28 provides maximum agitation of the liquid culture medium and specimen mixture 22 and the gas within each bottle 1. During a load or unload operation, door 13 is opened which allows to access approximately one third of all bottles 1 simultaneously. Then, drum 2 is rotated until the next third of bottles 1 becomes accessible. In three steps, all bottles 1 are accessible.

Alternatively, axis 28 of bell-shaped drum 2 is oriented vertically with a slight tilting of approximately 20 degrees away from door 13. By adjusting the tilt angle, the degree of agitation can be modified, if required, for maintaining optimum growth conditions.

In operation, bell-shaped drum 2 is rotated by motor 6 and a belt 7. A circular member 8 and a sensor 9 form an angular encoder that provides information about which row of bottles 1 is passing sensor station array 12. Preferably, motor 6 is a stepper motor, allowing drum 2 to rotate either in a continuous mode or to stop drum 2 at appropriate angles to read from sensing means 20 within bottles 1 in a steady-state mode. The whole system is controlled by a control system 10 located inside rotating drum 2. Output ends of all optical fibers 14 of the linear array of sensor stations 12 are fed to one common photodetector (not shown) in control system 10 such that only one excitation light source 11 needs to be turned on at a time. Therefore, the control system “knows” from which sensing station 15 and, therefore, which bottle 1 the sensor light is being collected.

The apparatus shown in FIGS. 1 and 2 positions the bottle to be detected by the sensor. However, solutions to any mis-alignment between sensor and bottle continue to be sought

### BRIEF SUMMARY OF THE INVENTION

Described herein is a system and method for detecting the position of sample containers (e.g. bottles) carried by a rotating drum (and changes thereto) in a blood culture processing apparatus relative to a measurement board disposed adjacent to the rotating drum. The systems and method of the present technology have a measurement and alignment module that may adjust at least one signal provided from a sensor of the measurement board based on the detected position of the rotating drum.

In one aspect, a system for detecting the position of a drum and the bottles carried in the drum is described. The system includes a drum-shaped rack, a measurement board, a plurality of timing targets, a target sensor, and a controller. The drum has an exterior perimeter and a plurality of receptacles that are each configured to receive a blood culture bottle. The exterior perimeter is disposed about an axis of rotation of the drum. The plurality of receptacles are arranged in the drum as an array of receptacles. The array has receptacles disposed both vertically and horizontally and the vertically aligned receptacles form a column and the horizontally aligned receptacles form a row. The measurement board is disposed at a stationary position adjacent to the drum and comprises a column of sensors configured to interrogate a column of bottles in the drum that moves past the measurement board as the drum is rotated about the axis. The plurality of timing targets are disposed around the exterior perimeter of the drum that rotate with the drum. The target sensor is disposed at a stationary position adjacent to the drum and is configured to detect one or more features of the timing targets as each timing target moves past the target sensor when the drum is rotated about the axis. The controller is configured to determine a position of the drum based on data from the target sensor.

In one aspect of the system, the determined position of the drum comprises a drum offset and a drum angle.

In one aspect of the system, the controller is configured to detect the position of the drum based on timing data associated with the detected features of the timing targets moving past the target sensor when the drum is rotated.

In one aspect of the system, the timing data comprises timing ratios associated with the plurality of timing targets.

In one aspect of the system, the controller is configured to normalize the timing ratios.

In one aspect of the system, the controller is configured to fit the normalized timing ratios to a sine function.

In one aspect of the system, the controller is configured to calculate the drum offset from an amplitude of the sine fit of the normalized timing ratios.

In one aspect of the system, the controller is configured to calculate the drum angle from a phase of the sine fit of the normalized timing ratios.

In one aspect of the system, the target sensor is an optical sensor.

In one aspect of the system, the optical sensor is configured to change states when a feature of the timing target interrupts a light path of the optical sensor.

In one aspect of the system, the controller is configured to detect the position of the drum based, at least in part, on state changes of the optical sensor.

In one aspect of the system, the one or more features of each timing target comprise a first edge and a second edge of the timing target.

In one aspect of the system, the first edge extends radially from the exterior perimeter of the drum and the second edge includes a first end and a second end, wherein a radial distance from the exterior perimeter of the drum to the second edge continuously decreases from the first end of the second edge to the second end of the second edge.

In one aspect of the system, the first end of the second edge connects to the first edge.

In one aspect of the system, the second end of the second edge connects to a first edge of an adjacent timing target of the plurality of timing targets.

In one aspect of the system, the plurality of timing targets are each aligned vertically with a column of receptacles of the drum.

In one aspect of the system, the controller is configured to adjust at least one signal of at least one sensor in the column of sensors in the measurement board based on the determined position of the drum.

In one aspect of the system, the at least one signal is a signal stored in a memory of the system.

In one aspect of the system, the drum has an upper end and a lower end and the plurality of timing targets are disposed proximately to the lower end of the drum.

In one aspect of the system, the target sensor is mounted to the measurement board.

In one aspect, a method for detecting the position of a drum and the bottles carried in the drum is described. The method includes: rotating a drum-shaped rack having an exterior perimeter about an axis of rotation of the drum, the drum having a plurality of receptacles, each receptacle configured to receive a blood culture bottle, wherein a plurality of timing targets are disposed around the exterior perimeter of the drum that rotate with the drum; accumulating sensor signals of a column of sensors of a measurement board that is disposed in a fixed position adjacent to the drum, the column of sensors configured to interrogate columns of bottles in the drum that move past the measurement board as the drum is rotated about the axis; accumulating data from a target sensor disposed at a stationary position adjacent to the drum, the target sensor configured to detect features of the timing targets as each timing target moves past the target sensor when the drum is rotated about the axis; storing the accumulated sensor signals and accumulated target sensor data in memory; calculating a position of the drum relative to the stationary measurement board based on the stored target sensor data from the target sensor; and adjusting at least one signal from the stored sensor signals based on the calculated position of the drum.

In one aspect of the method, the calculated position of the drum comprises a drum offset and a drum angle.

In one aspect of the method, the position of the drum is calculated based on timing data associated with the detected features of the timing targets moving past the target sensor when the drum is rotated.

In one aspect of the method, the timing data comprises timing ratios associated with the plurality of timing targets.

In one aspect of the method, the method further comprises normalizing the timing ratios.

In one aspect of the method, the method further comprises fitting the normalized timing ratios to a sine function.

In one aspect of the method, the method further comprises calculating the drum offset from an amplitude of the sine fit of the normalized timing ratios.

In one aspect of the method, the method further comprises calculating the drum angle from a phase of the sine fit of the normalized timing ratios.

In one aspect of the method, the target sensor is an optical sensor.

In one aspect of the method, the optical sensor is configured to change states when a feature of the timing target interrupts a light path of the optical sensor.

In one aspect of the method, the position of the drum is calculated based, at least in part, on state changes of the optical sensor.

In one aspect of the method, the one or more features of each timing target comprise a first edge and a second edge of the timing target.

In one aspect of the method, the first edge extends radially from the exterior perimeter of the drum and the second edge includes a first end and a second end, wherein a radial distance from the exterior perimeter of the drum to the second edge continuously decreases from the first end of the second edge to the second end of the second edge.

In one aspect of the method, the second end of the second edge connects to a first edge of an adjacent timing target of the plurality of timing targets.

In one aspect of the method, the plurality of timing targets are each aligned vertically with a column of receptacles of the drum.

In one aspect of the method, the drum has an upper end and a lower end and the plurality of timing targets are disposed proximately to the lower end of the drum.

In one aspect of the method, the target sensor is mounted to the measurement board.

### BRIEF DESCRIPTION OF THE DRAWINGS

FIG. 1 shows a front-view of the interior of a blood culture apparatus for the detection of microorganisms of the prior art;

FIG. 2 shows a side-view of the interior of a blood culture apparatus of the prior art;

FIG. 3A and FIG. 3B illustrate perspective views of a blood culture apparatus housing for the module described herein;

FIG. 4 is a top view of an incubation and measurement module according to one aspect of the present technology;

FIG. 5A-FIG. 5C illustrate the bottle rack drum with and without a section removed to reveal the bottle detection status indication (BDSI) board, in side views and a partial top view;

FIG. 6A-FIG. 6E illustrate the lift mechanism for servicing the motor in the drum interior;

FIG. 7A-FIG. 7D illustrate the BDSI board, the measurement board, and the controller connection board forming part of the measurement board and the alignment flags and section identifier sensor flags that work with the alignment sensors and the section identifier sensors on the controller board;

FIG. 8 illustrates the configuration of the drum position sensor that indicates to the BDSI board what drum section is aligned with a door to the drum module;

FIG. 9 illustrates one example of a progression of drum/bottle status panel lines patters used to direct a user to align the drum with the BDSI board;

FIG. 10A-FIG. 101 illustrate aspects of the light sources and detectors for bottle interrogation;

FIG. 11 is the drum illustrated in FIG. 5 with transparent internally reflective light pipes forming receptacles for receiving the blood culture bottles;

FIG. 12A-FIG. 12C illustrate a light pipe used in the drum of FIG. 11;

FIG. 13 illustrates top view of the bottle drum as describe herein;

FIG. 14 illustrates a bottle in a receptacle with a light pipe;

FIG. 15A-FIG. 15E illustrate a bottle retained in the receptacle with the light pipe;

FIG. 16 is a block diagram of a measurement system for detecting the position of a rotating bottle drum relative to a measurement board according to aspects of the present technology;

FIG. 17 is a partial side cross-sectional view of a rotating drum and measurement board, and an optical switch and a plurality of timing targets of the measurement system of FIG. 16;

FIG. 18 is a bottom view of an optical switch and a plurality of timing targets of the measurement system of FIG. 16;

FIG. 19 is a detail view of the optical switch and a portion of the plurality of timing targets of the measurement system of FIG. 16;

FIG. 20, FIG. 21 and FIG. 22 are partial perspective views of the rotating drum, measurement board, optical switch, and timing targets of FIG. 17;

FIG. 23 is a graph illustrating a sine fit of distance measurement data from normalized drum position for determining changes to the position of the drum;

FIG. 24 illustrates a flow chart for detecting changes in drum position and measurement of sample bottles, and adjusting sensor signals obtained from sample bottles by the measurement board;

FIG. 25, FIG. 28, and FIG. 31 each illustrate a radial plot of the position of a rotating drum under various loading conditions;

FIG. 26, FIG. 29, and FIG. 32 each illustrate a graph of a relationship between the percentage signal change of a measurement of the measurement board and a change in distance between a bottle in the drum and a sensor on the measurement board;

FIG. 27, FIG. 30 and FIG. 33 each illustrate a graph comparing an unadjusted sensor signal and a sensor signal that has been adjusted based on detected changes in drum position;

FIG. 34 is a partial side cross-section view of a rotating drum and measurement board, and first and second optical switches and plurality of timing targets of the measurement system of FIG. 16 in accordance with another aspect of the present technology;

FIG. 35 illustrates a timing target for use with the measurement system of FIG. 16 in accordance with another aspect of the present technology; and

FIG. 36 illustrates a flow chart for detecting changes in drum position and adjusting sensor signals obtained from sample bottles by the measurement board.

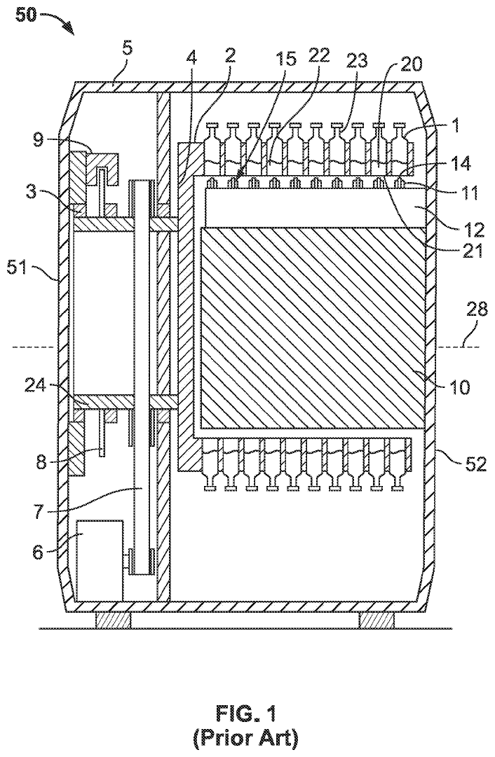

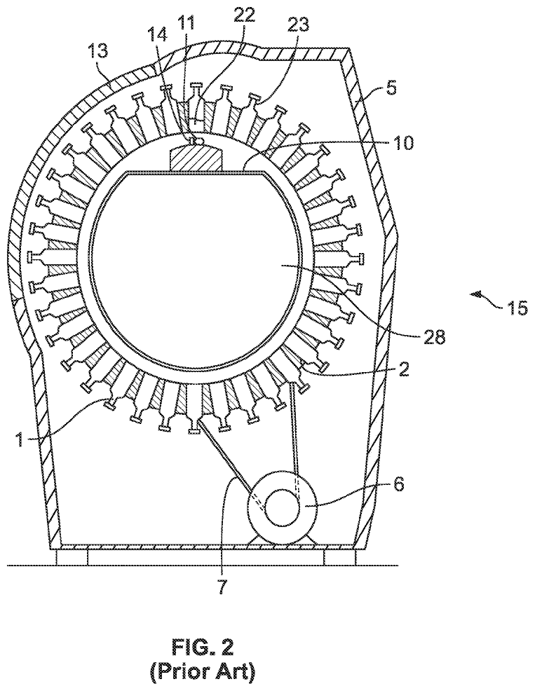

### DETAILED DESCRIPTION

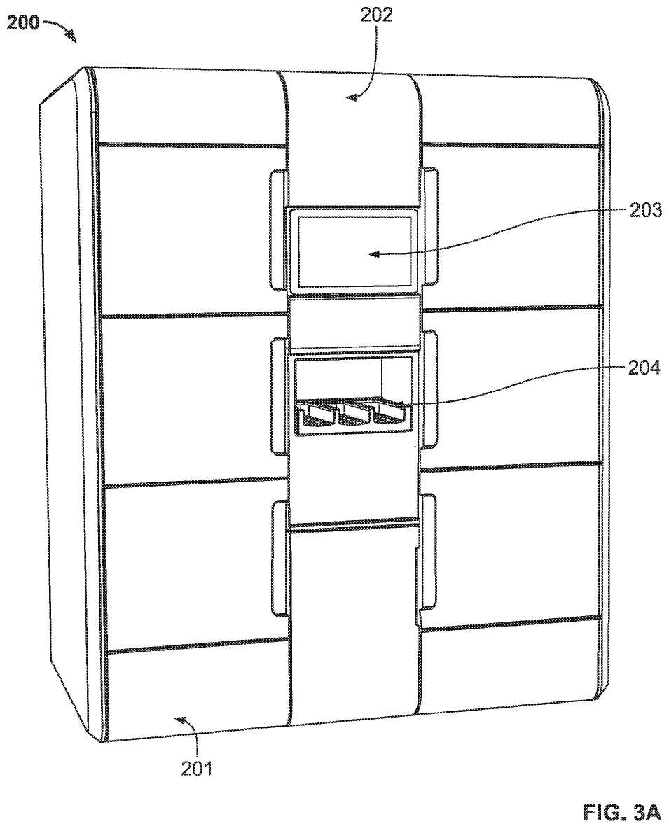

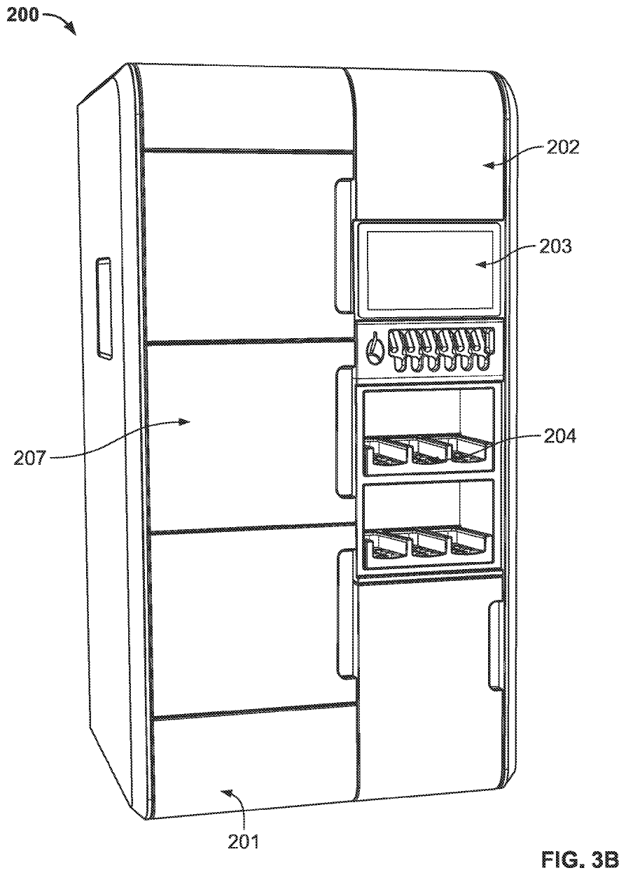

Described herein is a blood culture apparatus configured as an incubation and measurement module that, optionally, can be integrated with a larger end to end solution for processing biological samples to determine if such samples contain are contaminated with or infected by microorganisms. The module described herein can be placed in a cabinet such as is illustrated in FIGS. 3A and 3B. The cabinet 200 can provide power to the module, provide a controlled thermal environment to the module and a communication channel to the module. FIG. 3A illustrates a cabinet 200 with two three-door panels 201 that provide access to three bottle drums on each side of a central panel 202. Central panel 202 has touch screen 203 for data entry and used control. Central panel 202 also has a central station 204 for culture bottle input/output. FIG. 3B illustrates a cabinet with only one three-door panel 201.

Then number of doors will depend upon the number of drums. The number of drum racks in a module is largely a matter of design choice, with one-, two-, and three-drum configurations being contemplated herein. The apparatus described herein is not limited to any specific number of drums.

The module has a high-density bottle drum. High density, as used herein is a description of drum configurations that allow culture bottles to be placed closer to each other to allow a greater number of bottles to be fitted into the drum compared to the prior art. The module is configured to align bottles with a limited number of reader stations. That is, the number of reader stations is less than the number of bottle receptacles in the drum. Optionally, the drum is operated by a direct drive motor that can cause accelerated and decelerated drum movement (i.e. a rocking movement, intermittent rotation, etc.). A heater and blower are provided within the drum housing, or in spaces above or below the portions of the housing that receive the drums therein. The heater and blower circulate warm air around the drum. Optionally, the heater and blower will be configured to keep the temperature of the contents of all culture bottles in the drum within a predetermined narrow range of a specific target temperature. The predetermined narrow range is +0.5° C. of the target temperature. The specific target temperature is in the range of 30° C. to 40°. Optionally, the target temperature is 36.55° C. Greater temperature uniformity will permit an increase in set point as there is less risk of “over-heating” samples. A greater temperature uniformity at higher temperature will therefore permit a faster time to detection. The motor will permit the drum to be positioned such that the user or the automated apparatus can access any bottle carried by the drum. When the sample in a bottle is determined to be positive for microbial growth, a workflow is activated to retrieve that culture bottle from the module. The module is configured to assist with that workflow. The placement of module components such as the blower in the module are largely a matter of design choice, and not described in detail herein. The module may include other features such as vents, baffles, dampers etc. to further modulate and control the temperature and the temperature profile within the module.

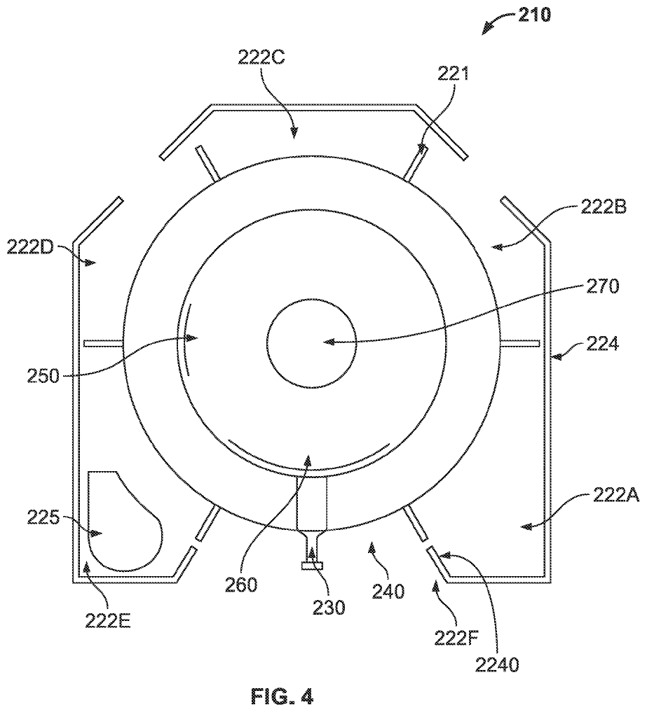

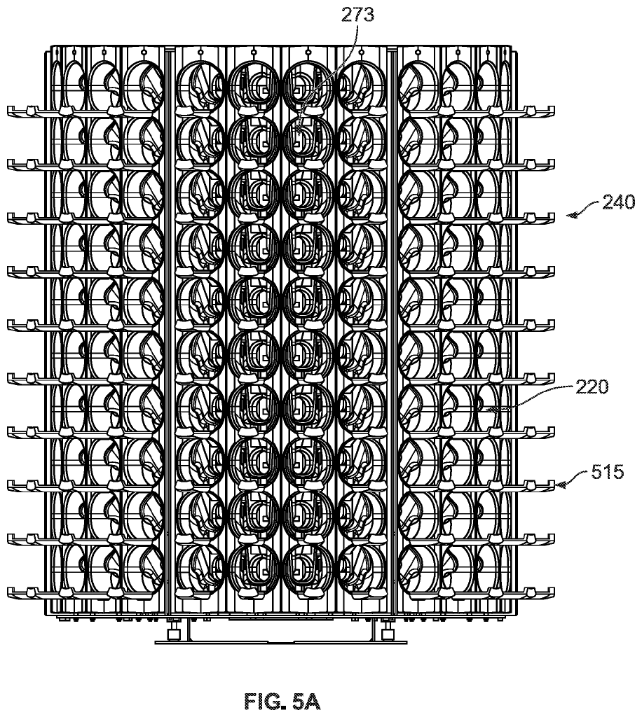

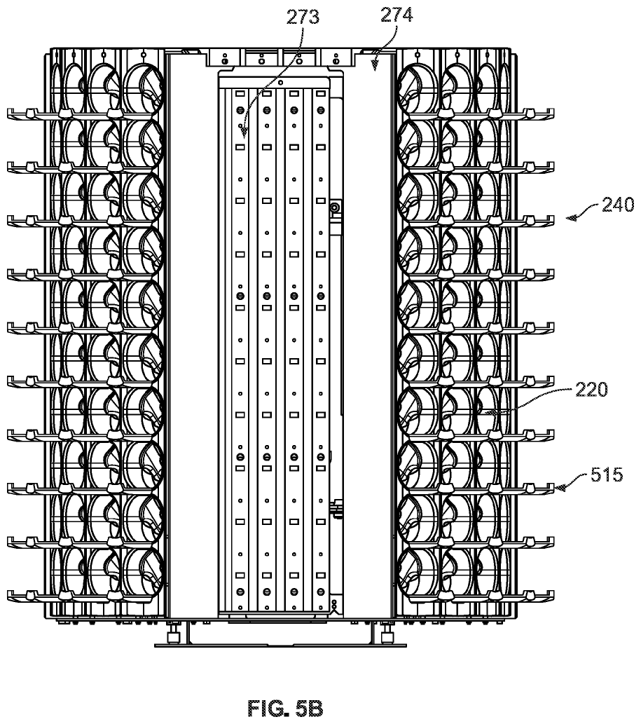

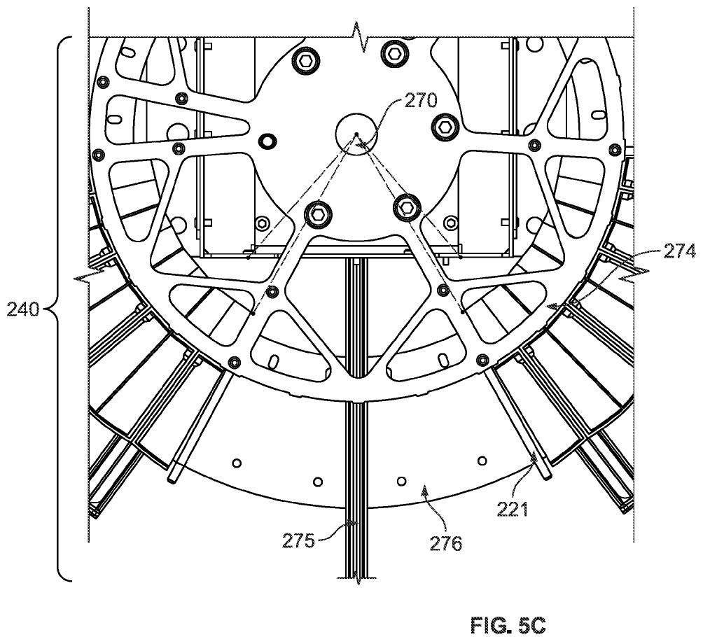

The module is configured to have LEDs and light pipes to indicate positive culture bottles to the user. Referring to FIG. 4, there is illustrated a top down view of an optional configuration of the module described herein. The module 210 has a housing 224, a blower and heater 225 for keeping the bottles 230 warm and a drum 240 with receptacles that hold the culture bottles. In the illustrated aspect, positioned in the interior of the drum are bottle presence sensor electronics 250 and culture bottle presence/status indicator electronics 260 (the BDSI board collectively includes these electronic components). A drive motor 270 is provided to rotate the drum 240. In other aspects described herein, the electronics are positioned adjacent the exterior of the drum. The drum 240 housing 224 has six panels 221 that define six drum sectors (222A-222F). As illustrated, about one-sixth of the drum contents (assuming the drum is full) are available for access at any given time since the span of one sector is about the same as the span of the opening in the housing through which bottles are added to or removed from the drum 240. FIG. 5A is a side view of the drum in FIG. 4. The culture bottles (not shown) are disposed neck inward in receptacles 220 in the drum 240 and received by cradles configured as a light pipe 515. The motor 270 is a direct drive motor provides high torque, little to no hysteresis, low noise, reliability and simplicity.

In one aspect, the drum 240 is configured such that the motor assembly 270 and the gearbox 271 are located in the interior of the drum 240. The drum 240 with receptacles 220 for receiving culture bottles (the culture bottles are not shown) is illustrated in FIG. 5A. As noted above, the drum 240 is assembled in sections of receptacles 220 and the drum 240 (with one section of receptacles 220 removed therefrom) is illustrated in FIG. 5B. A status indication board 273 that is more visible when the panel is removed is used to illuminate the light pipes 515 to indicate the status of the bottle cradled in the light pipe 515. The status indication (BDSI board) board 273 is also detachable and removable from the frame 274 of the drum 240. “Status” as used herein is the state of the blood culture bottle as determined by the module 210. The state of the blood culture bottle may be positive for microbial growth or negative for microbial growth. In one aspect, the status of a blood culture bottle is communicated by colored lighting of the bottle receptacle in the drum, with, in one aspect, green light indicating a bottle negative for microbial growth and red light indicating a bottle positive for microbial growth. The BDSI board 273 is also configured to indicate bottle presence in a station of the drum or if no bottle is present. The BDSI board 273 is also configured to indicate an error for a station.

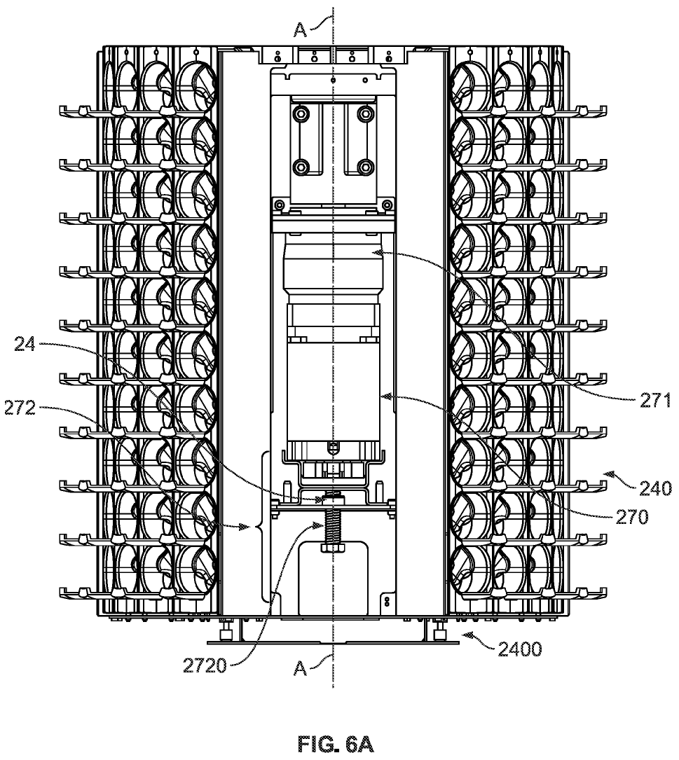

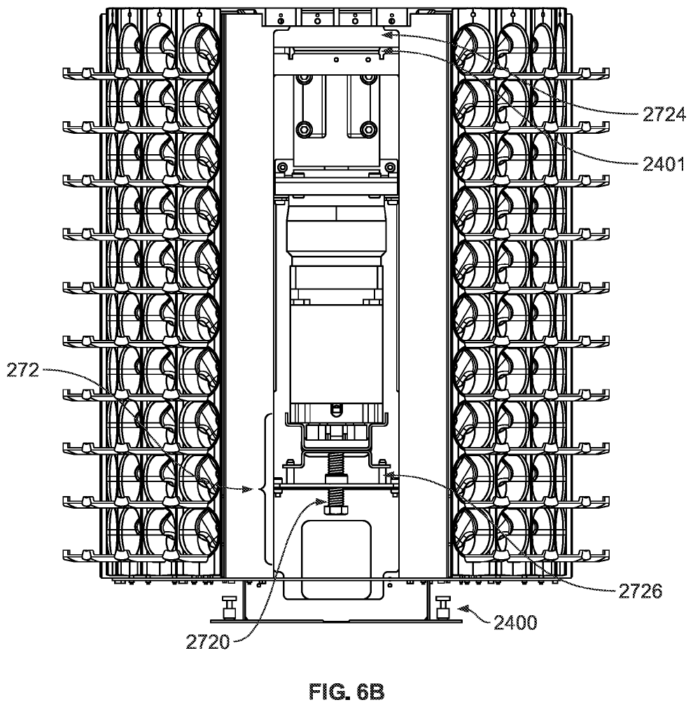

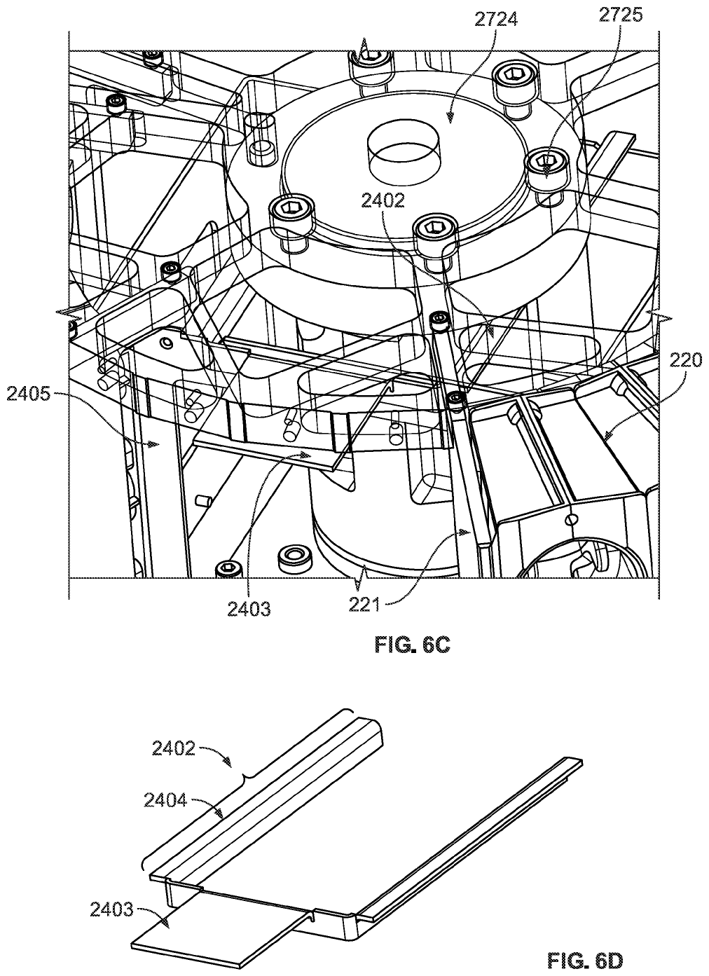

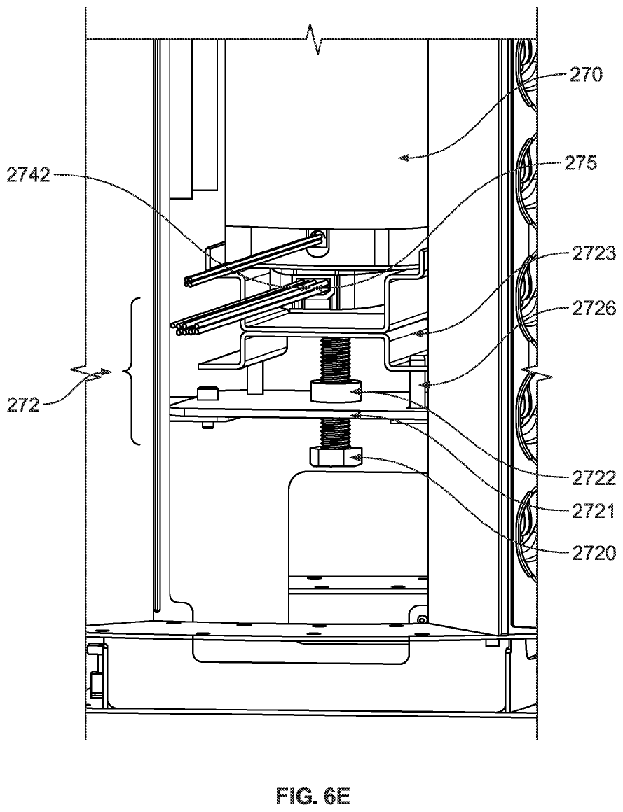

Referring to FIG. 5C, there is provided a top view of the frame 274 with the status indication board section removed therefrom. Cables 275 provide power to the motor assembly 270. Also illustrated is the bottom 276 of the frame 274. The drive motor assembly 270 drives the drum 240 to rotate axially. Bearings inside the gearbox of the drive motor assembly 270 provide both axial alignment of the drum 240 but also the requisite thrust load support required to advance the drum carrying a significant number of bottles 230. Referring to FIG. 6A, the axis A-A of the drive motor assembly 270 is aligned with the axis A-A of the drum 240. Consequently, the center of gravity of the drum 240/drive motor 270 assembly is on the center axis. The drum 240/drive motor assembly 270 is provided with a lifting mechanism 272 to expose the motor assembly 270 for service by lifting the drum 240. In one aspect, illustrated in FIG. 6E, the lifting mechanism is a screw 2720 that advances through motor assembly support plate 2721 and guide nut 2722. When advanced upward, screw 2720 forces plate 2723 upward. Plate 2723 travels upward along guide pins 2726. This raises the drum 240 off of drum supports 2400 and creates a lift space 2401 between the drum frame 274 and the rotor 2724 of the motor assembly 270. Referring to FIG. 6C, a locking tool 2402 is provided and is insertable into the lift space 2401 to lock the drum in place relative to the motor assembly 270 so that the motor can be serviced without carrying the weight of the drum 240. One example of a suitable locking tool 2402 is illustrated in FIG. 6D. The locking tool 2402, as illustrated has a handle 2403, affixed to a support bracket 2404. The locking tool 2402 is inserted between the rotor 2724 and the drum frame 274 (illustrated in phantom). The weight of the rotor 2724 and the drum frame 274 that is fastened to rotor via hex bolts 2725 is supported by service frame 2405 while the motor assembly 270 is being serviced. A detail view of the lifting assembly 272 is provided in FIG. 6E. Cables 275 provide power to the motor assembly 270. As explained previously, screw 2720 is advanced upward through plate 2721 and guide nut 2722 to raise support bracket 2723 (that travels along guide pins 2726) to lift the weight of the drum 240 off of the motor assembly 270 for service.

As previously noted, the apparatus is provided with a status indication board 273. As explained above with reference to FIG. 5A and FIG. 5B, the drum 240 is arranged in columns of receptacles 220, separated into sections, with multiple columns (e.g., four) in each section. In one aspect, one column in one section is reserved for reference bottles. User access to an individual section in the drum will be via a door that will give access to a single section (one of 222A-222F of receptacles 220) in the drum 240. The drum divided into sections is illustrated in FIG. 4.

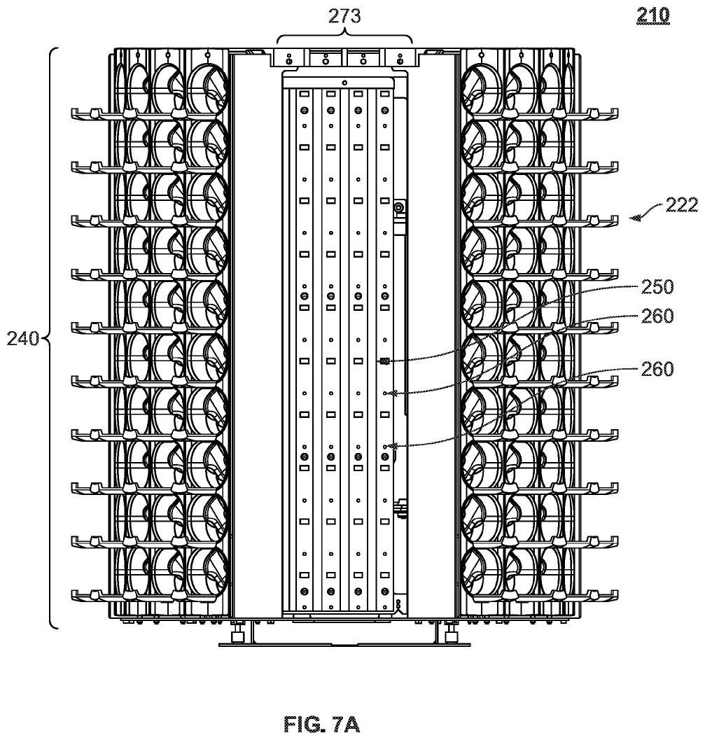

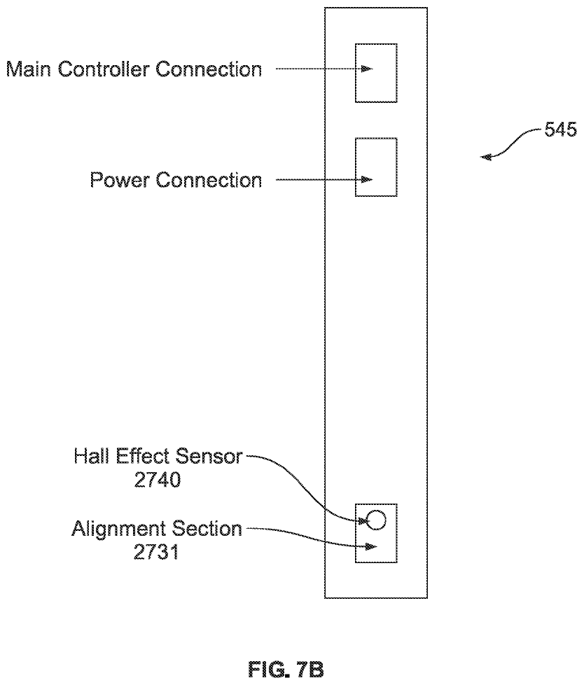

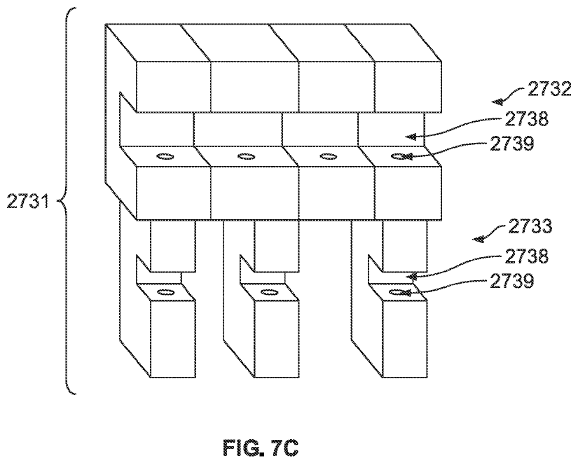

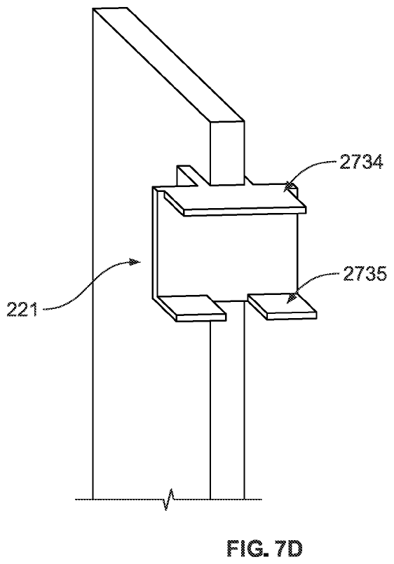

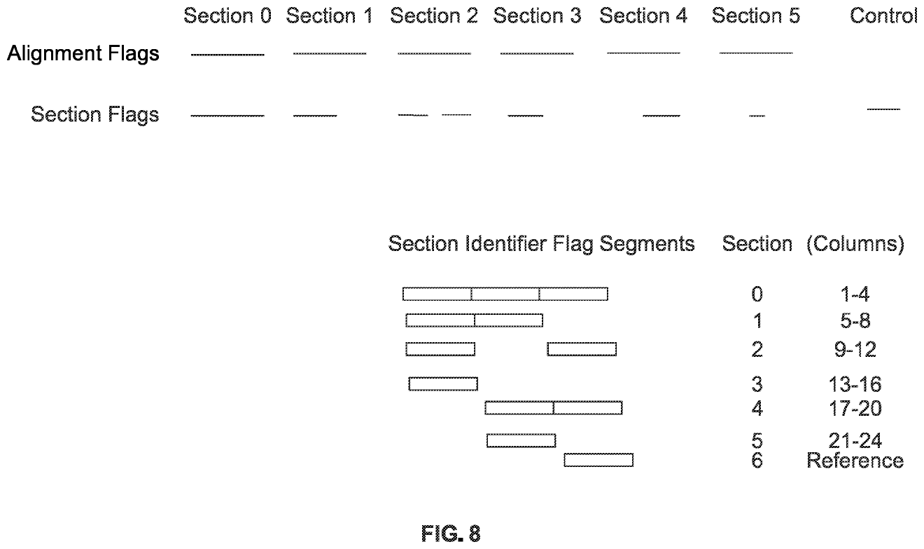

To sense bottle entry and removal, and provide feedback to the user during the user's access to sections 222A-222F in the drum 240, there are bottle presence sensor 250 (bottle sensors) and culture bottle/status indicator electronics 260 (e.g., lights) on the status indication board 273. See FIG. 7A. The bottle sensors 250 each sense when a bottle is inserted into or removed from receptacle 220 in the section 222A-222F that is accessible through the door to the drum 240. The indicator lights illuminate light pipes 515 in the drum receptacles 220, which transmit the light to be visible to a user viewing the receptacles through the open door. This requires that the light pipes 515 of the receptacles 220 be aligned with the culture bottle/status indicator electronics 260 in the status indication board 273. When aligned properly, the module 210 may positively indicate the status of the bottles (e.g., positive for microbial growth or negative for no microbial growth), and to detect when a bottle is inserted or removed.

To access all bottles in the drum 240, a user must cause the module 210 to rotate the drum. As each section of the drum 240 is turned into view through the opened door, the user may have a way to ensure that the exposed section (one of 222A-222F) of the drum 240 is aligned with the bottle presence sensors 250 and status indicator lights 260 of the status indication board 273. With reference to FIG. 7A, in one aspect, the module 210 may have an alignment mechanism (located on measurement board 545 or the BDSI 273) that serves to align the receptacles 220 in the drum 240 with the bottle presence sensors 250 and the status indicator lights 260 provided on the status indication board 273. One skilled in the art will appreciate that alignment can be accomplished in many different ways, and that the alignment mechanism is but one example of sensors that can be used to align the drum 240 with the module door and hence achieve alignment of the bottle receptacles with the bottle presence sensors 250 and the status indicator lights 260. Such alignment can be manual (e.g., the operator aligning the drum panels with the door opening); semi-automatic (a mechanism that the operator can control to advance the drum incrementally until alignment is achieved) or automatic (fiducials (e.g. the alignment flags) thar are detected and, based on their detected location, aligning the drum with the fiducial(s).

In one aspect, the alignment mechanism also includes alignment flags and section identification flags attached to the drum panels that separate the drum sections. A measurement board or BDSI may include an alignment section with optical sensors that detect the flags on the drum panels as they pass by the optical sensors as the drum is rotating is provided on measurement board 545. See FIG. 5B. BDSI 273 is positioned adjacent the drum interior, so that it can detect flags on the drum panels 221. The optical sensors are in communication with a main controller that, in one aspect of the module described herein, inform the user/operator of the drum section that can be accessed through the open door.

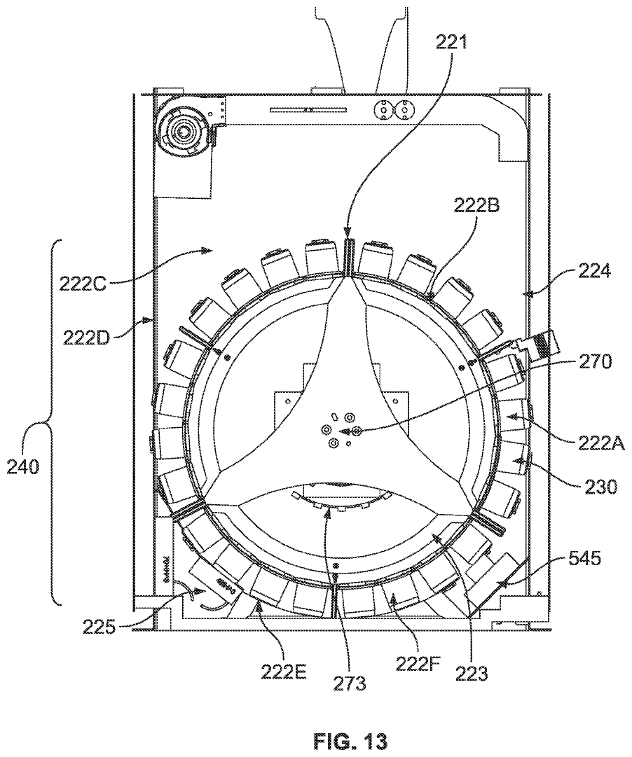

The status indication board 273 is also referred to as the bottle detection and status indication (BDSI) board 273. Status indication board 273 may be located behind the drum and aligned with the door opening as illustrated in FIG. 7A. As noted above, the module may also have a measurement board 545. In one aspect, the measurement board 545 is a controller board illustrated in FIG. 7B. With reference to FIG. 13, in one aspect, the measurement board 545 is fixed adjacent the exterior of the drum 240.

In one aspect, each column of the status indication board 273 may be either a single board or multiple interconnected boards. In the illustrated aspect, the four columns of the status indication board are connected to each other with, e.g., a flexible ribbon cable (not shown). The measurement board 545 is connected to a main controller board (not shown) for system communications. As illustrated in FIG. 7A, the status indication board 273 is mounted on the drum frame interior. By contrast, the measurement board 545, which may include an alignment section, is located on the drum exterior so that the flags 2734, 2735 may be detected by sensors 2739 on the measurement board 545.

Referring to FIG. 7C, the optional alignment section, 2731, may have a row of alignment sensors 2732 and a row of section identifier sensors 2733. In one aspect, the alignment sensors and the section identifier sensors may all be simple optical switches on the measurement board 545. As illustrated in FIG. 7C, the top row may contain four alignment sensors 2732 and the bottom row may contain the section identifier sensors 2733. The alignment sensors 2732, and the section identifier sensors 2733 may each have a notch 2738 in which a sensor 2739 (e.g., an optical switch) is disposed. Adjacent sections of the drum 240 may be separated by vertical panels 221 (also referred to as a drum rib, or wall) that extend out from the outer surface of the drum. Such are illustrated in FIG. 4 and FIG. 6C. As illustrated in FIG. 4, the drum panels or ribs 221 on either side of a section (222A-222F) align with the fascia 2240 of the housing 224 for a clean look when the user has the door open. The drum ribs 221 also extend to the inner surface of the drum as illustrated in FIG. 5C.

Each drum rib 221 inside the drum will have multiple flags (2734, 2735), one flag 2734 for drum alignment, and another set of one or more flags 2735 below the drum alignment flag 2734 for section identification. Flags 2734 and 2735 are detected when they pass through the notch 2738 and in the respective alignment 2732 and section identifier sensors 2733.

As noted above, each panel or rib 221 carries two flags, an alignment flag and a section identifier flag. As can be seen in FIG. 7D, the alignment flag may be a continuous flag to span across all four of the alignment sensors 2732. When the drum rotates such that all four of the optical switches in the alignment sensor 2732 register the presence of the flag, this indicates that the drum is in alignment. In one aspect, the sensors have an optical beam that transmits through the sensor gap. When the flag enters the gap, the optical beam is interrupted and this interruption registers as an indication of the presence of the flag. Although the flag interrupts the signal, this is referred to as the “on” state of the sensor, since, in this state, the sensor detects the presence of the flag. The section flags 2735 are configured as a unique identifier of a particular section of the drum. Consequently, the size and number of section flags varies for each section, so that each section creates a signal unique to its particular section. As illustrated, the sensors 2739 are positioned in a respective notch 2738 through which the alignment flags/section identifier flags pass. The sensors are configured as an emitter/receiver pair, only one of which is illustrated in FIG. 7C. In normal operation, the signal from emitter to receiver is uninterrupted. When the flags pass through the sensors, they “break” the optical beam and this provides an indication that the flags are present in the sensors. For a positive indication of alignment, the alignment flag must break the optical pathway for all of the alignment sensors. At that precise point, the number of signals “broken” for the section sensors will indicate which section is about to be rotated into position. Referring to FIG. 8, the number of sensors “fired” by the section flags passing through identifier sensors may inform the control software which section is present at the sensor location. At the point when the alignment flag 2734 is fully activating all alignment sensors 2732, the section identifier flags 2735 have activated the section identifier flag sensors 2733 by a wide margin. This will ensure the correct section of the drum 240 is identified when the alignment is correct. As stated previously, the alignment flag is the same size and configuration for each section, while the section flags each has a unique configuration such that the signal produced by a section flag indicates a particular section of the drum. As noted above, the alignment mechanism described above is one example of a mechanism that may be used to align the drum. One skilled in the art is aware of other suitable mechanisms for achieving alignment of the drum with the door and other module electronics (e.g., the status indication board 273).

The above arrangement allows a module controller (e.g. measurement board 545 or BDSI 273) to manage drum alignment without requiring communication with the main controller. The measurement board 545 may handle the process of helping the user align the drum to the bottle detection and status indication board (BDSI) once the module door is opened. The module will display the status of the stations whenever proper alignment is determined/indicated as described above.

The Main Controller may also handle the alignment of the drum prior to the door opening so the bottle status for the drum section visible when the user opens the door can already be lit when the user opens the door. [In one aspect, the Main Controller determines when a local controller (i.e. a controller in the module as opposed to the Main Controller) will activate the status lights on the status indication board 273. To commence a manual workflow, for example, the Main Controller may send a command to a module controller start alignment. From there, the Main Controller or the module controller may manage drum motion and status display. The local controller can be located anywhere in the module. In one aspect, the local controller is located on the measurement board 545. In another aspect the local controller may be located on the status indication board 273. The door to the module may be opened for a variety of reasons described herein. When the module door is closed, the status indicator lights are turned off. A command from the main controller may start alignment. When the door is to be opened, an alignment process commences and the drum is advanced until the alignment flag activates all of the alignment sensors mounted on the measurement board 545. When alignment is achieved, the local controller may also transmit to the main controller the specific drum section aligned with the door determined by the section sensor and the section flags detected when the alignment flag has activated the alignment sensor.

In one aspect, the main controller, in cooperation with and based on information from the status indication board 273, provides a status map for the module 210. The status map is updated when the user manually enters and removes bottles from the drum, or when bottles are added or removed by an automated apparatus in communication with the module 210. When the door to the module is to be opened, the main controller shares the status map with the status indication board 273. When bottles are added or removed from the module 210, the map is updated and shared with the main controller. The main controller may share the information with the command center if the module is not operating in isolation mode.

In one aspect, the local controller will enter an error state and indicate to a user that the module door must be closed. The conditions that might cause an error state are largely a matter of design choice, but can be something like a drop off in module temperature, a misalignment, etc.

As illustrated in FIG. 7A, the status indication board is equipped with a plurality of lights that may convey information to the user. Although located behind the drum 240 and the bottle receptacles 220, the lights convey information to the user through the light pipes 515 in the receptacles 220. In one aspect, the status indication board 273 may convey information as a pattern of light/light of different colors. The light patterns/colors/meanings are a matter of design choice. Examples of the information conveyed include the station/receptacle status (blocked, available, etc.) and bottle status (positive, negative etc.). The status displayed is communicated from the main controller to the local controller.

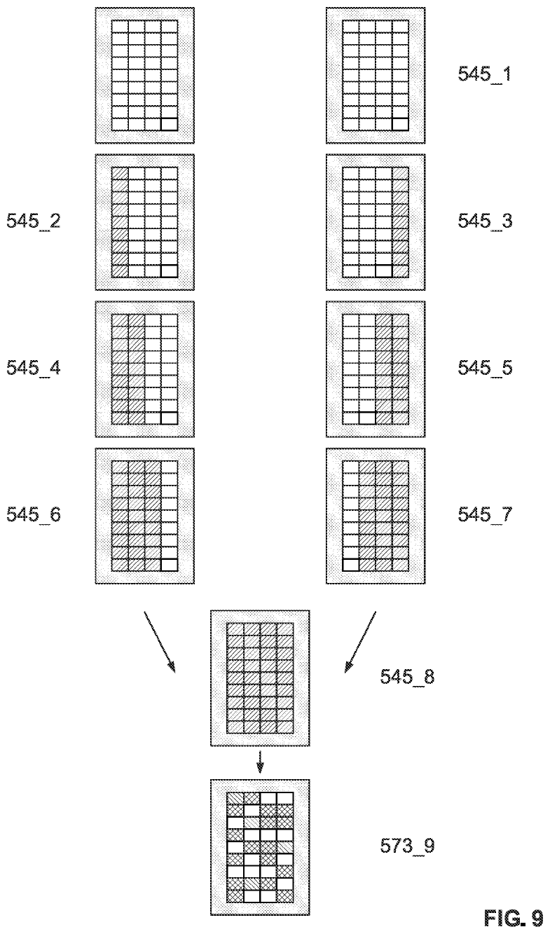

Measurement board 545 may be in communication with a panel that conveys alignment status to a user. FIG. 9 is one example for the progression of displayed alignment status with multiple lights for conveying information about the alignment status of the instrument. For example, when the alignment flags 221 are not in alignment with the alignment sensors 2731, the lights are all dark 545_1. As the drum section moves into alignment, the columns illuminate as the flags begin to activate the alignment sensors either moving from left to right (illuminating the left column 545_2 first) or from the right to the left (illuminating the right most column 545_3 first). This progression of the alignment flags through the alignment sensors (either from the left to the right or from the right to the left) are illustrated for detection of the alignment flag by the second alignment sensor (545_4; 545_5) or the third alignment sensor (545_6; 545_7). When all four alignment sensors detect the presence of the alignment flag, all alignment sensor indicator lights are illuminated (545_8). In another aspect, the panel will simply communicate no alignment (all lights off) or total alignment (e.g., all lights on).

Once all of the alignment sensors are lit, the status indicators change to the station statuses 273_9 and the user may begin manual operations such as placing bottles in or removing bottles from the module 210. The panel will still provide for an indication of alignment but will have some tolerance built in as the drum may move slightly during manual operations. This will avoid triggering a misalignment reading that might require a module reset. In one example, once complete alignment is achieved, alignment continues to be indicated as long as either the left most or right most alignment sensor continues to detect the presence of a flag. If there is no flag detected by any of the alignment sensors, then the local controller turns off all of the indicator lights and a new alignment protocol may be commenced.

Once alignment is confirmed, a panel 273_9 in communication with the status indication board 273 will indicate the status of the individual bottles in the receptacles. For example, two-way cross hatched lights indicate a sample negative for microbial growth (col. 1, row1, col. 2, rows 3 and 8, and col. 4, row 5). One-way cross hatched lights may indicate bottles positive for microbial growth (Col. 1, rows 2, 4, 6, 8 and 9; Col. 2, rows 1 and 5; Col. 3, rows 2, 3, 5, 6, and 8; and Col. 4, rows, 2, 7, and 9). The unlit lights indicate that no bottles are present at those locations. One advantage of the present design is that the user can advance the drum manually when the door is opened. To advance the drum automatically, it is advantageous to have the door closed so that nothing gets caught in the advancing drum. In one aspect, the drum may need to be powered off when the door is open. In that mode of operation, the drum may not be able to be advanced when the door is open.

In another aspect, rather than using light pipes to convey bottle status as described above in the context of FIG. 9, a BDSI panel may be provided on the exterior of the drum to convey the alignment information. The BDSI controller would be connected to the BDSI panel to illuminate the indicators, which could be light pipes, lenses etc.

As described above, the rotating drum rotates past the measurement board 545. Since the drum 240 rotates past these various detection devices on the measurement board, the measurements occur in what is described as a “fly-by” fashion, fly-by being the rotating drum moving the bottles past the measurement electronics as the measurements are being made. The measurements being made ascertain whether the blood culture bottles are positive or negative for microbial growth. As such, measurement sensors on the measurement board 545 are provided to interrogate the bottles to determine if their internal gas composition or pH is dynamic (i.e., changing) in a manner that is indicative of metabolic activity inside the bottle as a consequence of microbial growth. For example, bottles with a measured increase in carbon dioxide or a measured decrease in oxygen concentration over time may be determined to be positive for the growth of microorganisms. To make this determination, light sensors are directed at a chemical sensor in the bottle that is indicative of bottle conditions (e.g. oxygen concentration, carbon dioxide concentration, pH). The location of the chemical sensor in the bottle will depend upon what the sensor is measuring. The headspace in the bottle is the portion of the bottle interior where the gasses are separated from the liquids and solids in the blood culture (i.e., the sample, the nutrients, etc.).

Since a culture bottle may be interrogated multiple times before a determination is made that the culture bottle is positive or negative for microbial growth, the measurement conditions must be consistent enough measurement to measurement or an adjustment may need to made to the measurement in the instance of distance variability. This means that the light from the interrogation sensors 2501 and the distance from the bottle sensor to the photodiode detector 2602 should remain relatively constant measurement to measurement.

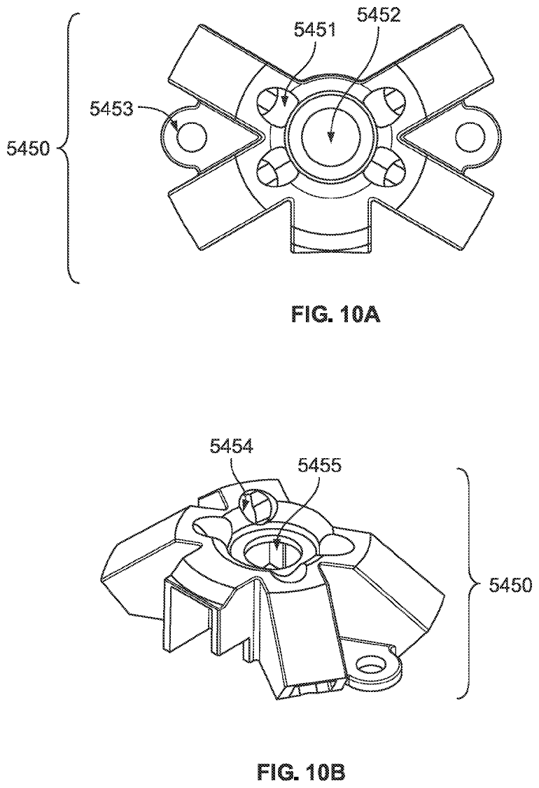

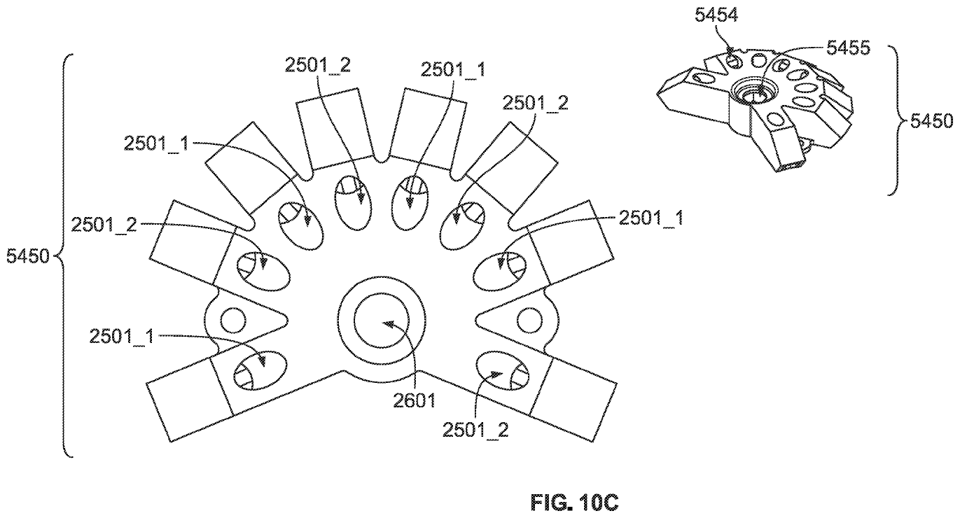

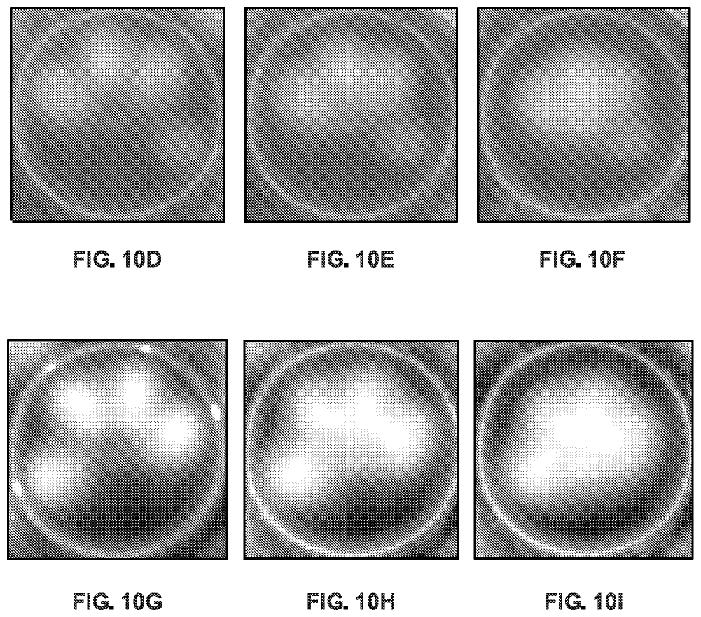

As described above, the bottles are interrogated on a column by column basis as the drum 240 with the rows of bottle receptacles 222 rotate past the measurement board 545. Referring to FIG. 10A-101, the sensors (e.g. the light sources 5451) and the detector 5452 are provided in a housing 5450 which is fastened to and extends from the measurement board 545. Light sources 5451 are positioned around a single light detector (photodiode 5452). The housing has a fastening mechanism (flanges 5453) for fastening the housing 5450 onto the measurement board 273. As illustrated in FIG. 10B, the housing 5450 has ports 5454, 5455 for receiving the light sources 25451 and for receiving the photodiode detector 5452. The ports 5454, 5455 are configured so that the light sources 5451 surround the photodiode detector 5452 and are angled towards the photodiode detector 5452 such that the light emitted by the light sources 5451 intersects above the photodiode detector 5452 on the bottle sensor (not shown) directly opposite the photodiode detector 5452.

As the distance between the bottle and measurement board 545 increases, the combination of the excitation light from the light sources 5451 spreading out, and the fluorescence it induces in the bottle sensor being farther from the photodiode detector 5452, cause the signal produced by the photodiode detector 5452 to decrease. The light sources 5451 may be located farther radially from the photodiode 5452 since the point of intersection of the light from the light sources 5451 relative to the bottle sensor is what provides measurement to measurement consistency. It is advantageous if the light emitted by the light sources 5451 intersects with the bottle behind the bottle sensor directly opposite the photodiode. The intersection point is far enough behind the bottle sensor, causing the illumination of the sensor to be off center such that the fluorescence induced by the light sources 5451 is partially out of the field of view of the photodiode 5452.

As the bottle moves away from the measurement board 545, the light from the light sources 5451 converges onto the center of the bottle sensor disposed in the blood culture bottle and therefore moves into the field of view of the detector 5452. This additional fluorescence of the sensor, caused by the additional light from the light sources impinging on the bottle sensor, offsets the decrease in fluorescence to be detected by the detector 5452 by virtue of the fact that the bottle, and the sensor in the bottle, are slightly further away from the detector 5452.

Referring to FIG. 10C, the housing 5450 has eight light sources 2501 therein. Four light sources are a first color and are designated 12501_1. The light sources are light emitting diodes (LEDs). In one aspect, four light sources are a second color and are designated 12501_2. In one aspect, the first color is green and the second color is cyan. As illustrated, the color of the light sources alternates around the photodetector 5452. The housing 5450, with the ports 5454 for receiving the light sources 5451 and the ports 5455 for receiving the photodiode detector 5452, is illustrated in the upper right. FIGS. 10E-10J illustrates the transition of light impinging on the bottle sensor (from closer to farther). FIGS. 10E-10G illustrate this transition for the cyan LEDs and FIGS. 10H-10J illustrate the transition for the green LEDs (again, from closer to farther). The above design mitigates the measurement to measurement variations that arise due to a measurement to measurement difference in the distance between the light sources/photodetector and the bottle sensor. The intensity of a diffuse light source drops proportionally to the square of the distance from the light source. This is why the photodiode signal produced by the fluorescence that results for the light from the light sources drops as the distance between the measurement board 545 and the bottle increases. Not only does the fluorescence intensity received by the photodiode decrease, but the intensity of the source light impinging on the sensor also decreases.

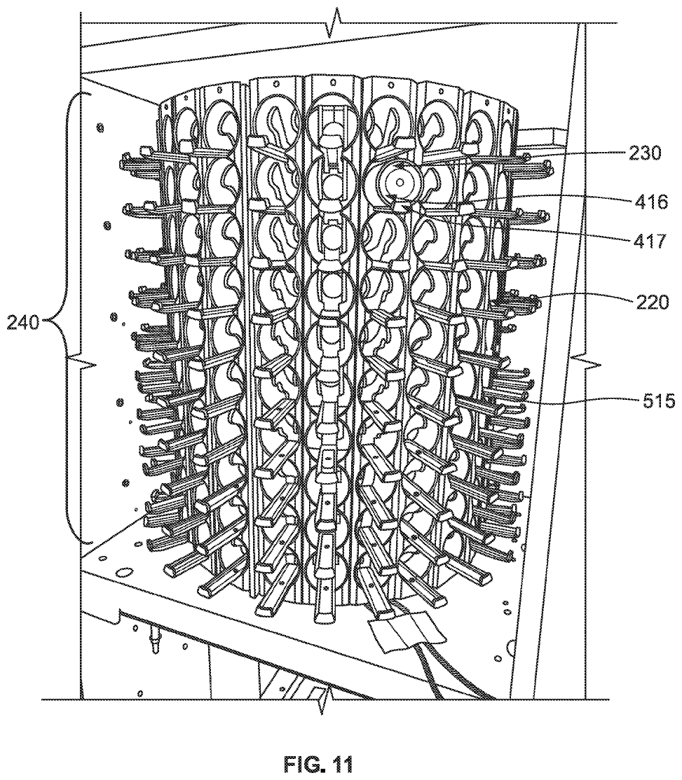

Referring to FIG. 11, the bottle drum 240 has receptacles 220 for receiving culture bottles disposed neck-in therein as described above. The receptacles 220 have a light pipe 515 formed in the bottom portion of the receptacle that also defines the bottom edge of the receptacles 220. The light pipes are formed from a material that will transmit light through the light pipe structure but prevent the light from emanating from the light pipe, to prevent appreciable cross-talk from an illuminated light pipe to an unilluminated light pipe. Examples of suitable material include polycarbonate (e.g., Makrolon 2258), and acrylic (e.g., polymethyl methacrylate). Makrolon® (formerly Hyzod®) is a trade name for Covestro (formerly Bayer MaterialSciences). These materials are all polycarbonate which is a very tough, high impact plastic material. Translucent materials are contemplated, but partially transparent light pipe materials may make perceiving the color of the light pipes more challenging.

Referring again to the status indication board 273, the LEDs that illuminate the light pipes 515 may be a plurality of LEDs that may illuminate the light pipe in a number of different colors, each indicating a different status of the blood culture bottle being held in the receptacle 220 that has the light pipe. In one aspect, the LEDs illuminating the light pipe are on the status indication board and about 5 mm from the light pipe. As described herein, once the culture bottle status is determined, the BDSI, in conjunction with the local controller or main controller, determines the color to illuminate the light pipe 515 (e.g., red for positive, green for negative, etc.). The light pipes are configured to both provide a color indication of bottle status and retain the culture bottle 230 in the drum receptacle 220. In this configuration, the culture bottle bottom 416 is secured in the receptacle by tab 417.

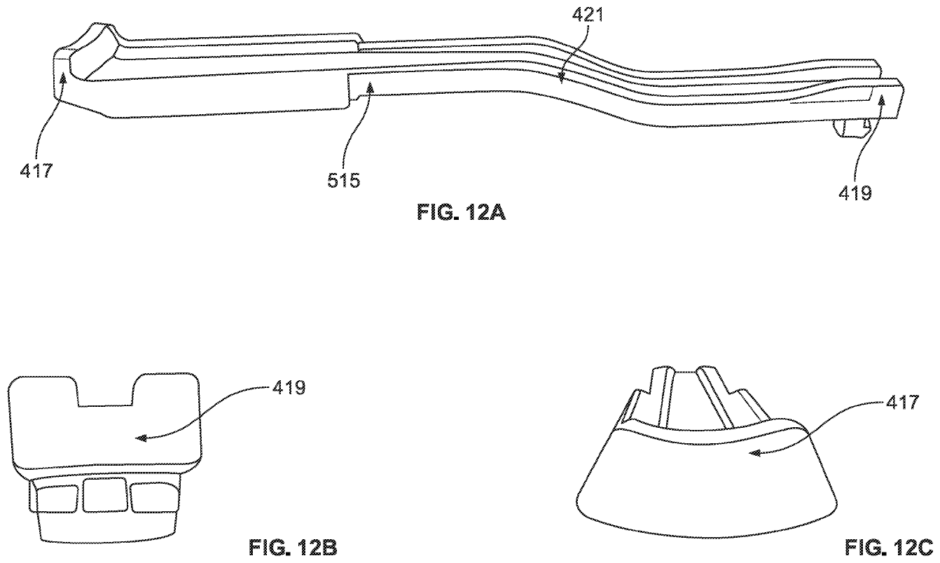

FIG. 12A is a perspective view of the light pipe receptacle 515 with the light entrance end 419 and the tab 417. The light pipe 515 is configured as a waveguide for the LEDs positioned at the light entrance end. As such, the light pipe is configured to have a total internal reflection to render it suitable as a waveguide. In one aspect, the light pipe 515 has a refractive index of about 1.52, which is higher than that of the surrounding air. In one aspect, light pipe is cladded and the refractive index of the waveguide portion of the light pipe 515 is higher than the refractive index of cladding on the light pipe 515. FIG. 12B is the light entrance end 419 and FIG. 12 C is the tab 417.

In order for the light pipe to have the requisite total internal reflection, any curves must be mild, with no sharp curves or dead corners. The mild curvature is illustrated as 421 in FIG. 12A by way of example. In one embodiment, the light pipe has a body length of about 110 mm from the light entrance end 419 to the tab 417. For purity of transmission, it is advantageous if the presence of foreign particles and bubbles is minimized. In one aspect, the light pipe is molded polyacrylate. While the use of 3D printing to form the light pipes is contemplated, it is easier to control the quality of light pipes formed by molding. In one aspect, the light pipe has very low, little or close to zero internal absorption, and is free of foreign particles and bubbles. For example, and not by way of limitation, low or little internal absorption is less than about 0.2 dB cm⁻¹.

Referring to FIG. 13, the bottles 230 (facing inward) in the drum 240 are placed into one of six sectors (222A-222F) in the module 210. The sectors are demarcated by vertical panels 221 that extend outwardly from the drum 240. The span between adjacent panels is approximately equal to the span of a door into the housing 224 to completely shield the user from the inside of the module when a section of culture bottles is being accessed by the user. The module 210 also includes a blower and heater 225 for keeping the bottles 230 warm. Positioned in the interior of the drum is the status indication board 273. A drive motor assembly 270 is provided to rotate the drum 240. The measurement board 545 is positioned on the outside of the drum and takes fly by measurements of the bottle and drum flags to determine bottle status and drum alignment, respectively.

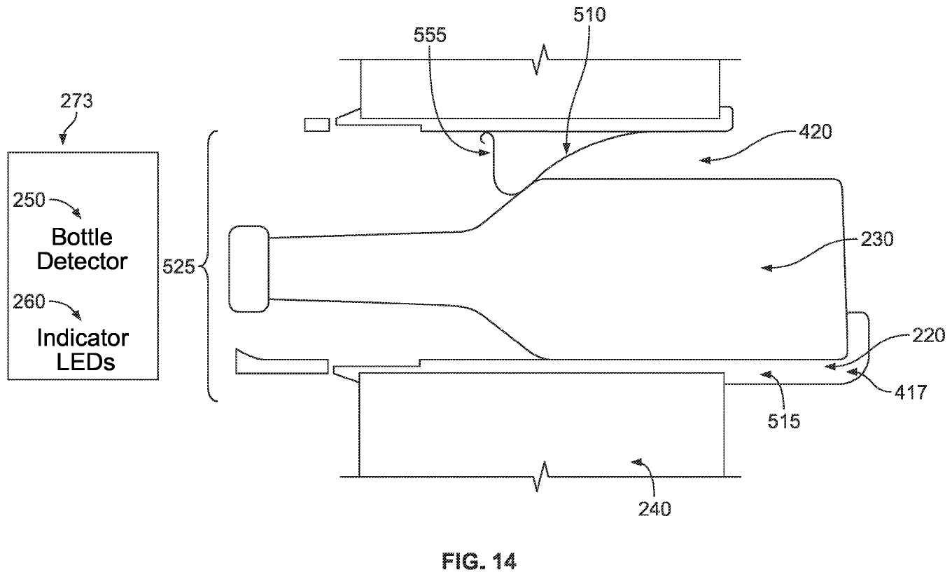

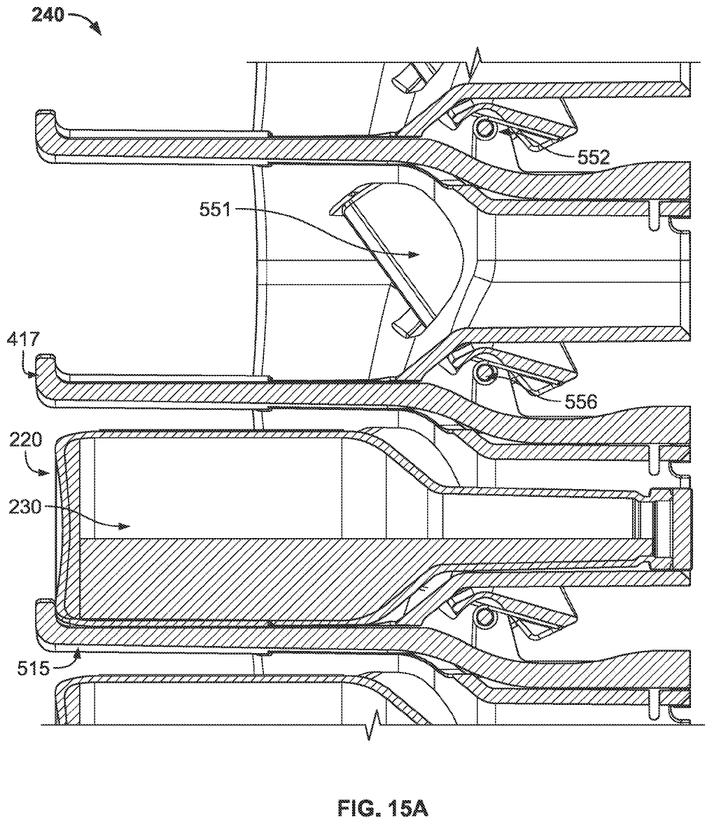

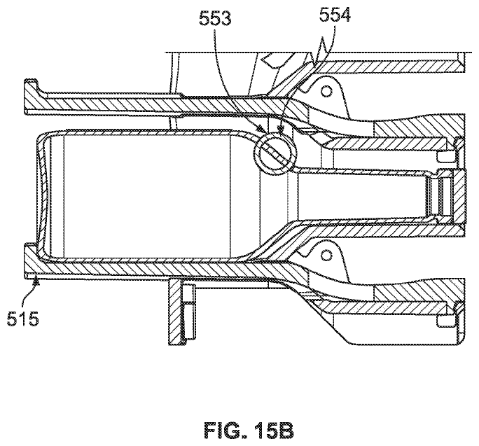

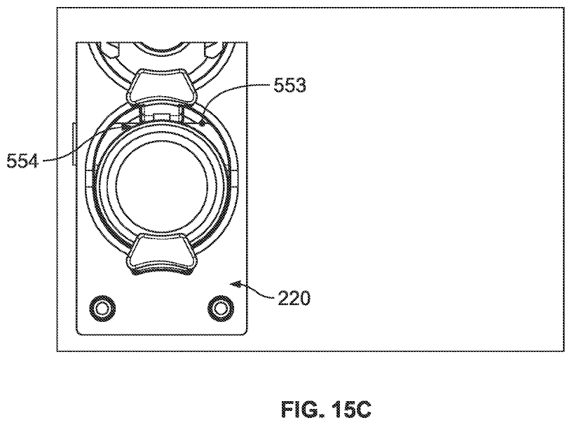

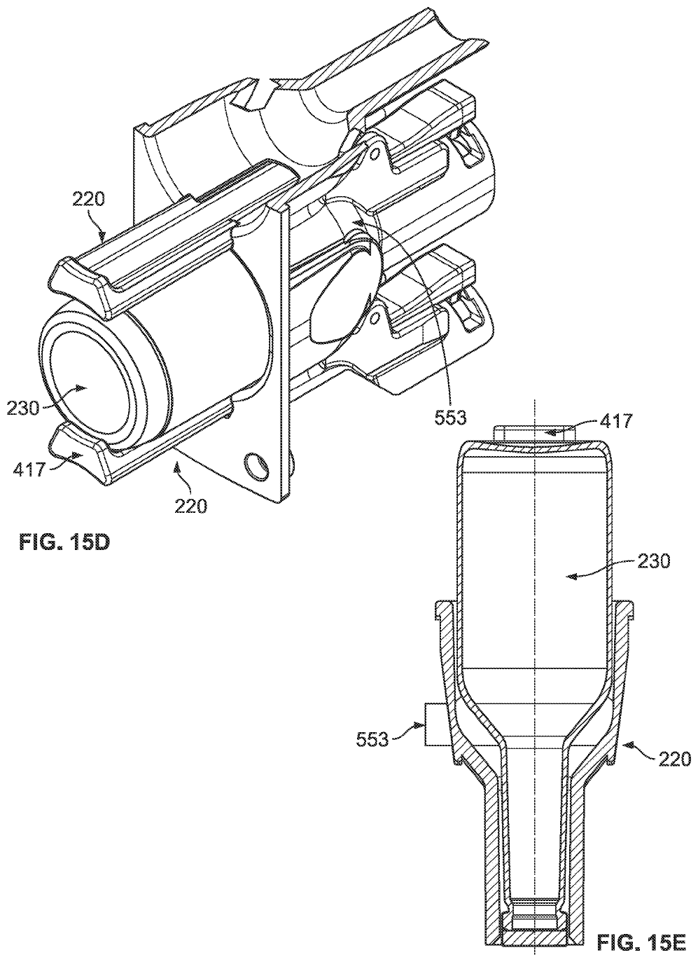

With reference to FIG. 14, the receptacle is illustrated with the light pipe 515 to translate the light from indicator LEDs 520 from the distal end 525 of the receptacle (i.e. the inside of the drum 240 in which the receptacle is disposed). The light pipe 515, if present, cradles the bottle 230 and extends past the proximal end 420 of the receptacle (i.e. the outer surface of the drum 240). The flat spring 510 presses against the upper shoulder 535 of the culture bottle 230 to hold the culture bottle 230 against the tab 417. The receptacle 220 is adjacent to a bottle presence detector 250 at the distal end 525 of the receptacle 220. The distal end of a bottle 230 in receptacle 220 is detected by bottle presence detector 250. The bottle presence detector 250 is carried by the status indication board 273. As illustrated in FIGS. 15A-15E, the spring is enmolded into the bottle holder 220.

The light pipe 515 lines up with indicator LEDs 260 on the status indication board 273. The surface of the end of the light pipe 515 outside the bottle drum 240 is textured to disperse the light from the indicator LEDs 260. The bottle crimp cap 410 interrupts the bottle presence detector 250 (e.g., an optical switch or a proximity sensor) when it is placed in the receptacle 220. The indicator LEDs 260 and bottle presence detector 536 are located on the status indication board 273 that is positioned on the inside of the drum 240 in an arrangement that corresponds to each bottle 230 in the drum 240 that is accessible by the user. The bottle presence detectors 250 are monitored while a door to the module is open to detect in real time when a bottle 230 is placed in a receptacle or removed from a receptacle.

FIG. 15A illustrates, a portion of a drum 240, with multiple vertical rows of the receptacles 220. The drum is illustrated in cutaway view to show the culture bottle 230 support in receptacles 220. The top receptacle 220 is empty. In the embodiment illustrated in FIG. 15A, a pivoting arm 551 is provided to secure the culture bottle 230 in the receptacle 220 instead of the leaf spring 550 previously described. As the bottle 230 is advanced into the receptacle 230, the pivoting arm 551 rotates clockwise to secure the culture bottle 230 in the receptacle. Resistance to pivoting arm 551 is applied by coil spring 552, which is secured in the receptacle with a pin 556.

An alternative to the receptacle illustrated in FIG. 15A is illustrated in FIG. 15B to FIG. 15E. Referring to FIG. 15B, the pivoting arm 551 illustrated in FIG. 15A is replaced by a deformable material 553. In one example, the deformable material 553 is a peristaltic tubing but other conventional deformable materials are contemplated. A key aspect of the deformable material is its resilience in assuming its undeformed shape after each instance of being deformed by insertion of the culture bottles in the receptacle. The bottom portion of the receptacle 220 is light pipe 515.

The deformable material 553 is placed in tapered portion 554 of the receptacle 220. Referring to FIG. 15C, an end view of the receptacle (220) illustrates the deformable material 553 at the top portion of the receptacle, (along the tapered portion 554 of the receptacle). Suitable deformable materials include, in addition to the elastomeric peristaltic tubing described above, elastomeric materials and foam materials.

FIG. 15D is perspective view of one receptacle 220 with a portion of second receptacle formed above it. The culture bottle is retained in the receptacle as described above. Tab 417 retains the culture bottle 230 in the receptacle 220. Other materials for the deformable material are contemplated as having sufficient frictional properties when used in contact with bottle 230. Such friction prevents the rotation of bottle 230, thus allowing the measurement system to obtain high quality signals with less noise caused by bottle vibration due to rack movement. FIG. 15E is a top down view of the culture bottle retained in the receptacle 220 with the light pipe 515.

As explained herein, the module rotates the drum both for positioning the bottles for user access and also for automation access. The module also rotates the culture bottles to agitate them.

The apparatus according to the present technology described above provides at least the following advantages: 1) a reduction of noise (i.e., the ratio of growth signal to reference signal should be unaffected by bottle position, temperature, and sensor variability); 2) detection of growth in a vial that experiences a delay in entry into the system (i.e., the dual measurements described above provide a reference such that the contents of the vial do not need to be sampled continuously during growth to confirm positivity by detecting growth acceleration); and 3) signal quality indicator (i.e., the reference signal is an independent indicator of the health of the station hardware).

As described above, the fly-by reading/measurement technique employed in relation to drum 240 and measurement board 545 relies on a relatively constant or fixed distance measurement to measurement between the bottles 230 in receptacles 220 of the drum 240 and the column of sensors on measurement board 545 for measuring and interrogating the bottles 230. Variations in distance and position of the drum 240 relative to measurement board 545 affects the distance between bottles 230 and the corresponding sensors on measurement board 545 and thus affects the fluorescent readings from the media bottles. Such variations in position and distance from measurement to measurement may cause noise in the readings of the bottles. Thus, the readings obtained from measurement board 545 may be inaccurate during the presence of these variations, if not corrected.

In one aspect, a fully loaded drum may weigh 80-90 lbs. and spin at 30 rpm. The drum 240 may change orientation slightly, e.g., wobble, when unbalanced by changing bottle loading and rotated for (fluorescent) measurements. The change in orientation or position of drum 240 relative to board 545 will also cause variations in distance between the bottles in drum 240 and the corresponding sensors of measurement board 545. Such variations in distance will cause the fluorescent bottle signal to change (e.g., decrease in strength as the bottle distance from the corresponding sensor on measurement board 545 increases and vice versa). Such changes in distance may result if the drum axis tilts slightly from its normal position.

Below, systems and methods for detecting changes to the distance between the bottles and the measurement board that may result from changes in the position or orientation of the drum are described in accordance with aspects of the present technology. Moreover, systems and methods are described for offsetting or compensating or adjusting the readings obtained by the measurement board in response to detected variations to distance and position. It is to be appreciated that the aspects of the systems and methods described below may be implemented and used with any of the aspects described above in relation to the blood culture apparatus including drum 240 and measurement board 545.

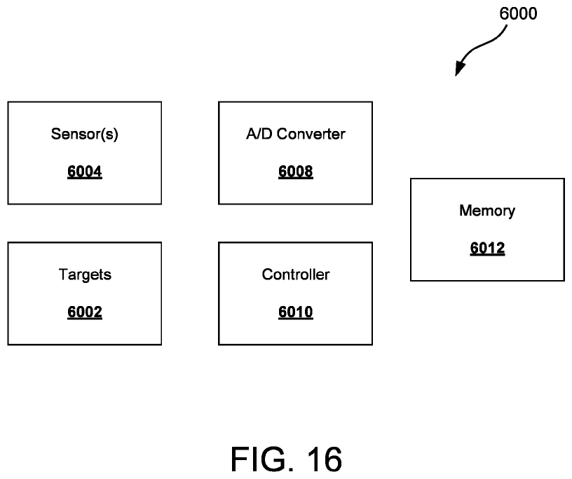

For example, referring to FIG. 16, a block diagram of a measurement system 6000 for detecting the position or orientation of a rotating drum of a blood culture apparatus relative to a stationary measurement board of the blood culture apparatus is shown in accordance with an aspect of the present technology.

System 6000, as shown, includes a plurality of timing targets 6002 disposed around the exterior perimeter of the drum, sensor(s) 6004, analog-to-digital (A/D) converter 6008, controller 6010, and one or more memory 6012. It is to be appreciated that the components of system 6000 may be discrete from the blood culture apparatus components or may represent one or more components of the blood culture apparatus described above. For example, controller 6010 may be any of the controllers or modules described in relation to drum 240, measurement board 545, etc., described above, or a discrete controller of system 6000.

As will be described in greater detail below, the timing targets 6002 may be disposed at discrete locations on (or proximate to) the exterior perimeter of the drum. In one aspect, the timing targets are deployed as a continuous series of timing targets along the exterior perimeter of the drum. Sensor(s) 6004 for detecting (features of) the timing targets 6002 as they pass by the sensor 6004 may be positioned at a location adjacent to the drum (e.g., on the measurement board adjacent to the drum) such that the timing targets 6002 pass by and/or through the sensors 6004 during rotation of the drum. The readings from sensor(s) 6004 may be converted by the A/D converter 6008 from analog to digital readings for use by controller 6010 to detect a position or orientation of the drum (and changes thereto) and/or determine changes in distance between each bottle in the drum and a corresponding sensor of the measurement board fixed adjacent to the rotating drum from measurement to measurement. Sensors 6004 may also measure the timing of the passing of the timing targets 6002 by or through the sensors 6004, indicating changes in distance. Controller 6010 (or another controller of the apparatus) is configured to use the changes in distance and/or position to adjust one or more of the stored readings obtained by the sensors on the measurement board relating to the bottles in the drum.

In one aspect, during a test cycle, system 6000 is used to determine initial relative positions of a set of calibrator and reference bottles in some or all bottle locations in the drum, and sensors in various positions on the stationary measurement board adjacent to the drum, along with fluorescent readings from the calibrators and reference bottles in the drum while in those initial relative positions. This information is recorded in a memory, such as memory 6012. Any subsequent changes to the initial relative positions recorded can be determined or calculated by controller 6010 (using measurements obtained from sensor(s) 6004 in relation to targets 6002) to adjust the stored fluorescent readings from each location in the drum such that changes in the adjusted readings are due only to changes in the bottle and not changes in position of the drum relative to the stationary measurement board.

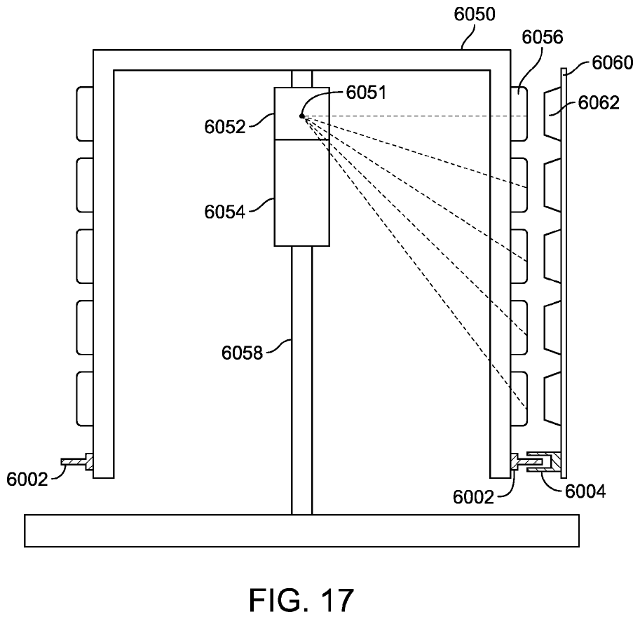

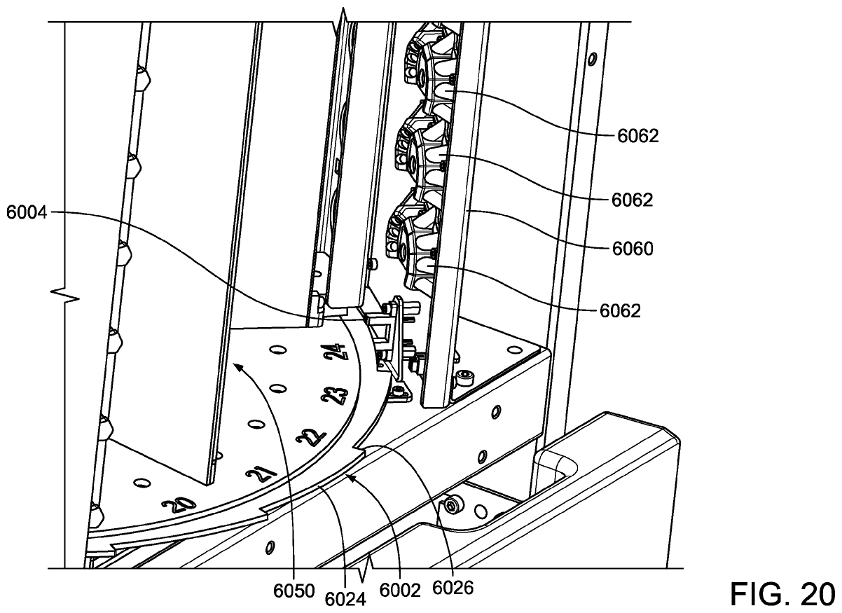

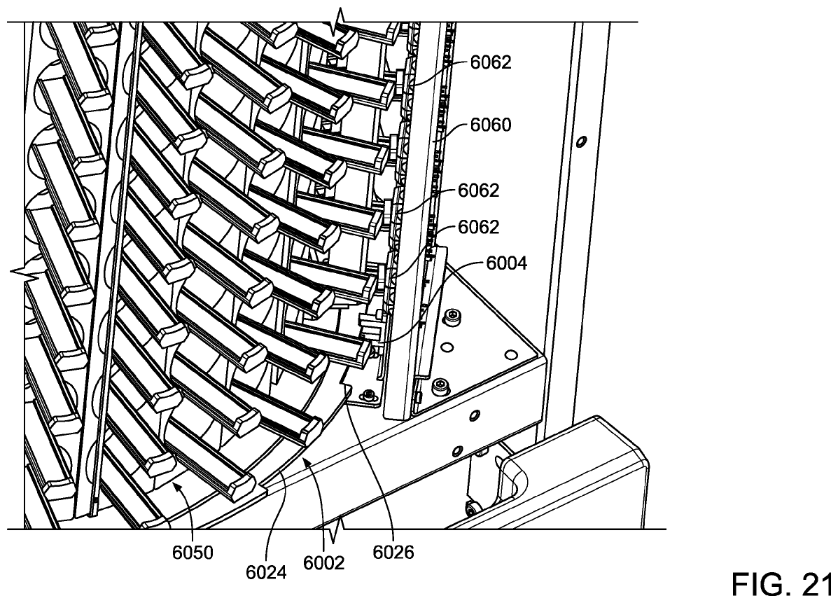

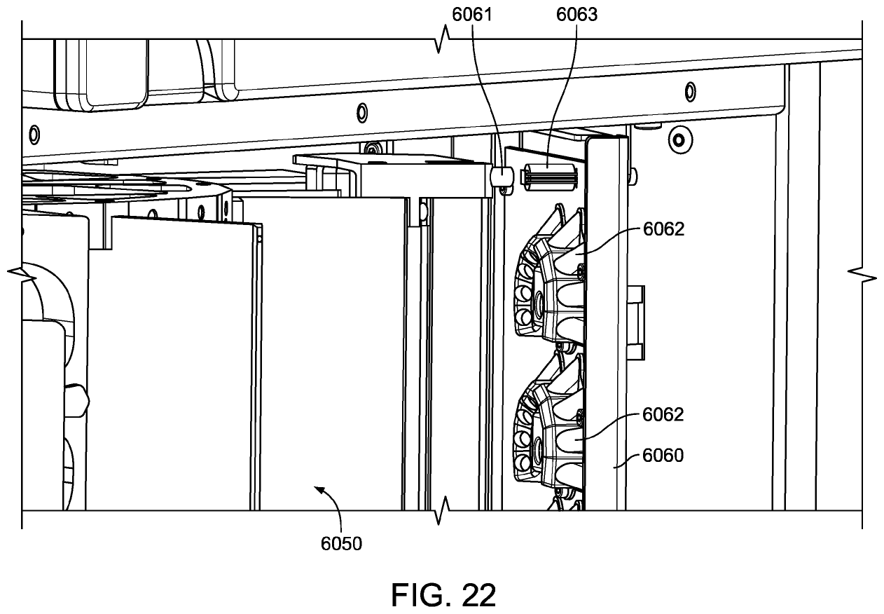

Referring to FIGS. 17, 20, 21, and 22, a blood culture apparatus including a drum-shaped rack 6050 (drum hereinafter), measurement board 6060, and measurement system 6000 is shown in accordance with aspects of the present technology. It is to be appreciated that drum 6050 may include any of the features of drum 240 described above and measurement board 6060 may include any of the features of measurement board 545 described above.

The drum 6050 includes columns and rows of bottles 6056 held in an array of receptacles arranged around the exterior perimeter of the drum. A motor 6054 and a gearbox 6052 are attached to the frame of the drum 6050 via a shaft 6058. The motor 6054 and gearbox 6052 cooperate to rotate the drum 6050 such that respective columns of bottles 6056 held in receptacles of the drum 6050 fly or pass by a measurement board 6060 including a column or set of sensors 6062 for reading the bottles 6056 as the column of bottles passes the column of sensors 6062.

The plurality of targets 6002 are mounted to the outer perimeter of the drum 6050 and extend radially therefrom. The targets 6002 are disposed at discrete locations around the outer perimeter of the drum 6050. For example, a target 6002 may be placed at a lower end of the drum 6050 at each column of receptacles in the drum 6050 or at each vertical section divider of the drum 6050. One skilled in the art will appreciate that targets 6002 may be placed at various discrete locations around the drum 6050.

The position or orientation of the drum 6050 may be defined by three variables. The first variable may be a drum offset, which is the distance of the center of the drum from the center of motion, at the level of the targets 6002. The second variable may be the angle of the maximum drum offset relative to the home position of the targets 6002 and drum 6050. The third variable may be the pivot point of the drum, which is the position of the point that constrains the movement of the drum. The pivot point position is measured relative to the targets 6002. An unbalanced drum (e.g., due to asymmetrical loading of the drum receptacles with bottles 6056) will wobble around the pivot point. In one aspect, the pivot point for drum 6050 is a fixed pivot point 6051 in gearbox 6052. The drum 6050 can only pivot around pivot point 6051. It is to be appreciated that the pivot point for the drum will depend on the construction of the drum assembly and is known from the assembly's configuration. The position of any bottle 6056 (or receptacle containing the bottle) in the drum 6050 can be calculated based on the angle and offset described above and the position of the pivot point 6051 above the targets 6002.

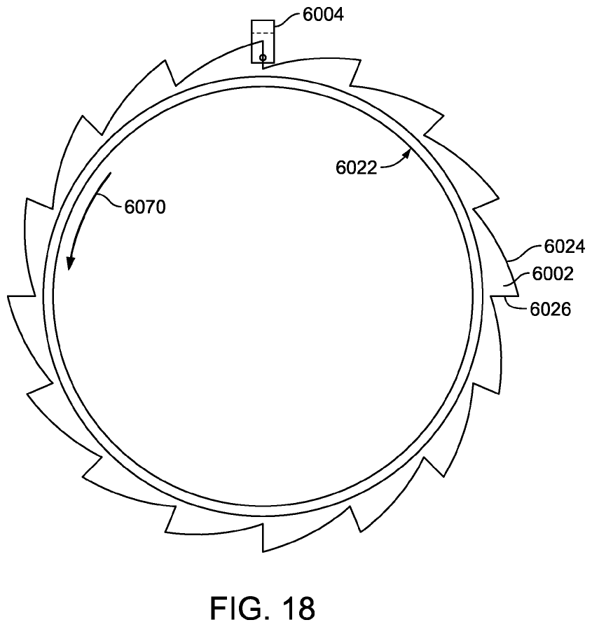

As shown in FIG. 18, in one aspect, a continuous series of the plurality of targets 6002, extend radially from an outer circumference of a target ring base 6022 mounted to or integrated with the drum 6050.

As shown in FIGS. 17 and 20-22, the inner circumference of the ring base 6022 is shaped to be received by a portion of drum 6050, for example, at a lower edge of drum 6050 that is opposite to gearbox 6052 and motor 6054. Ring base 6022 is mounted to drum 6050, such that, drum 6050 and targets 6002 are rotated together in unison.

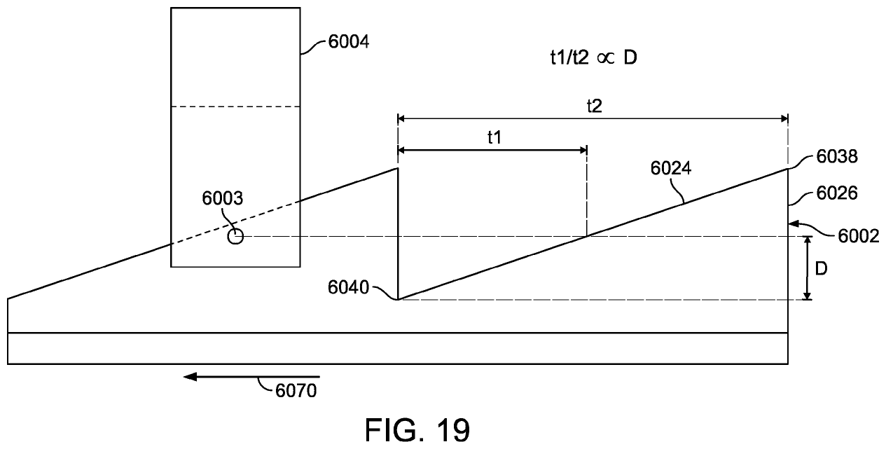

In one aspect, sensor 6004 may be an optical switch with a light path 6003 (shown in FIG. 19). Switch 6004 is mounted in a fixed position at a location selected such that, when drum 6050 and targets 6002 are rotated, at least a portion of each target 6002 passes through the light path 6003 of switch 6004, which is held fixed relative to the drum movement in one aspect. As shown in FIGS. 17 and 20-22, in one aspect, switch 6004 may be mounted to the bottom of the measurement board 6060. In another aspect, switch 6004 may be mounted in a fixed position separate and apart from measurement board 6060. For example, switch 6004 may be mounted to a surface of the cabinet that drum 6050 is disposed in such that at least a portion of each target 6002 passes through the light path 6003 of switch 6004.

Referring to FIGS. 18 and 19, in one aspect, each target 6002 includes a radial edge 6026, which extends radially away from the exterior of drum 6050 and is aligned with the center of the drum. Each target 6002 further includes an edge 6024 that includes a first end 6038 that meets an end of radial edge 6026 and a second end 6040 that, in one aspect, meets the radial edge 6026 of an adjacent target 6002. In another aspect, there may be some distance between the second end 6040 of a first target and the radial edge 6026 of an adjacent target. In any case, from the first end 6038 to the second end 6040, the radial distance of the edge 6024 from the exterior of the drum continuously decreases. It is to be appreciated that edge 6024 may be curved, as illustrated in FIG. 18, or may be straight, as illustrated in FIG. 19.

As drum 6050 rotates in rotational direction 6070, the targets 6002 move through the light path 6003 of switch 6004. In one aspect, switch 6004 is configured to be in a first state or a second state depending on whether the light path 6003 is blocked or unblocked. In one aspect, when no portion of a target 6002 is blocking light path 6003, switch 6004 is in a first state (e.g., an open state). When any portion of a target 6002 passes over or blocks light path 6003, switch 6004 is in a second state (e.g., a closed). Thus, as drum 6050 moves in rotational direction 6070, the state of switch 6004 changes from closed to open when some portion of edge 6024 passes through light path 6003 and the state of switch 6004 changes from open to closed when edge 6026 passes through light path 6003. In this way, the state changes of switch 6004 indicate the passage of edges 6024, 6026 through light path 6003.

Controller 6010 is configured to monitor the state changes of switch 6004. In one aspect, system 6000 includes an A/D converter 6008 configured to receive the changing states of switch 6004 as analog output signals and convert the analog signals to digital signals that are provided to controller 6010 and/or memory 6012. In one aspect, switch 6004 is monitored by controller 6010 during A/D readings at a clock rate of approximately (e.g., +/−10%) 1 kHz. It is to be appreciated that this clock rate is exemplary and the present disclosure contemplates other suitable clock rates. Controller 6010 is configured to maintain a counter (e.g., in software stored in memory 6012 and executed by controller 6010 or in a discrete processing component of system 6000) that is incremented by controller 6010 at each clock cycle. The number of clock cycles between state changes of switch 6004 are recorded into memory 6012 by controller 6010.

From the state changes of switch 6004 and the number of clock cycles between state changes, controller 6010 is configured to determine the amount of time that switch 6004 is open or closed and the time elapsed between the detection of each of edges 6024 and 6026 as a target 6002 passes through switch 6004. The time elapsed from switch open to switch close from switch 6004 is proportional to the position of drum 6050 and the distance between drum 6050 and measurement board 6060.

It is to be appreciated that the depth or radial length of the radial edge 6026 of each target 6002 determines the range of motion of the drum 6050 that can be measured. Thus, the radial length of radial edge 6026 is selected based on the known range of motion or variation in distance between drum 6050 and measurement board 6060. In one aspect, the radial length of each of the radial edges 6026 are selected to be approximately (+/−10%) 5 mm, however, it is to be appreciated that other radial lengths for radial edge 6026 are contemplated.

In one aspect, controller 6010 is configured to determine that a radial edge 6026 of a target 6002 is detected when the state of switch 6004 changes from the open state to the closed state. Moreover, in this aspect, controller 6010 is configured to determine that an edge 6024 of a target 6002 is detected when the state of switch 6004 changes from the closed state to the open state.

In one aspect, controller 6010 may be configured to use the alignment and/or section flags described above for determining which target 6002 has passed through a switch 6004. For example, as shown in FIG. 22, in one aspect, the drum 6050 includes a home flag 6061 mounted thereon and the measurement board 6060 includes a home flag sensor 6063 mounted thereon and configured to detect when home flag 6061 passes. The first radial edge 6026 of a target that is detected after the home flag 6061 is detected by home flag sensor 6063 represents a 0° drum angle of drum 6050 and identifies the target 6002 associated with that position of the drum 6050. Data from, or representative of, the readings of sensors 6063 and 6004 are provided to controller 6010 such that controller 6010 can use the data to detect or determine the drum angle at a given time. In this regard, each radial edge 6026 is associated with a drum rotational angle relative to the home position. Thus, by counting the number of radial edges 6026 detected after the home flag 6061 is detected, controller 6010 may detect the drum angle at that time. For example, as described above, in one aspect, detection of the first radial edge 6026 after home flag 6061 is detected represents a 0° drum angle of drum 6050 and identifies the target 6002 associated with that position of the drum 6050, detection of the second radial edge 6026 after home flag 6061 is detected represents a 22.5° drum angle of drum 6050 and identifies the target 6002 associated with that position of the drum 6050, detection of the third radial edge 6026 after home flag 6061 is detected represents a 45° drum angle of drum 6050 and identifies the target 6002 associated with that position of the drum 6050, etc. In this way, the controller 6010 is configured to determine the position of each target 6002 and the drum angle at the time that each target 6002 passes through switch 6004. It is to be appreciated that the angular spacing between targets 6002 and the drum angle that each target represents are known (or stored and accessible via memory 6012) by controller 6010.

In one aspect, controller 6010 is configured to determine times t1 and t2 shown in FIG. 19 based on the elapsed time between state changes of switch 6004 and use the ratio of t1/t2 to determine changes in distance between drum 6050 and optical switch 6004. The distance between drum 6050 and optical switch 6004 may then be used to determine the distance between drum 6050 and measurement board 6060.

Time t1 is the time elapsed between a time instant that the radial edge 6026 of a preceding target 6002 (e.g., the left most target in FIG. 19) is detected via the state change (e.g., open to closed state change) of switch 6004 and a time instant that edge 6024 of a successive target 6002 (e.g., the right most target in FIG. 19) is detected via the state change (e.g., closed to open state change) of switch 6004.

Furthermore, time t2 is the time elapsed between a time instant that the radial edge 6026 of the preceding target 6002 is detected via the state change (e.g., open to closed state change) of switch 6004 and a time instant that the radial edge 6026 of the successive target 6002 is detected via the same state change (e.g., open to closed state change) of switch 6004. In other words, t2 is the time that it takes for entire length of target 6002 along the rotational direction of drum 6050 to pass through switch 6004.

In one aspect, controller 6010 is configured to calculate the times t1 and t2 from the number of clock cycles recorded between the state changes of switch 6004 as each of the targets 6002 pass through switch 6004. The ratio of t1/t2 is calculated by controller 6010 for each target 6002. The ratio is proportional to the distance between the optical switch 6004 and the exterior of the drum 6050 and is used by controller 6010 to determine the position or orientation of the drum 6050. If it is determined that the position of drum 6050 is changed (or the distance between the bottles in drum 6050 and the sensors on board 6060 have changed), the stored measurements obtained by the sensors of measurement board 6060 may adjusted by a controller (e.g., controller 6010, a controller of measurement board 6060, or another controller of the blood culture apparatus) to compensate for such changes.

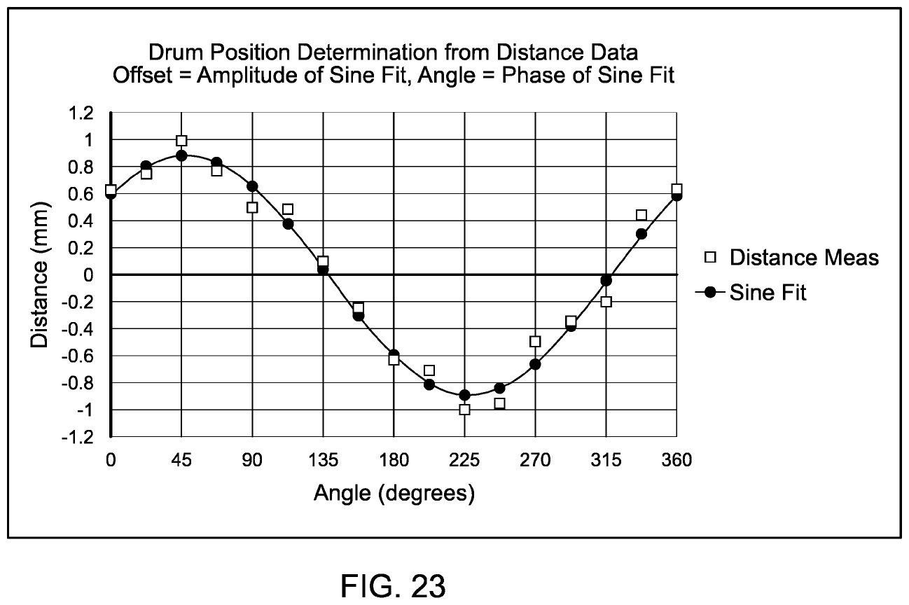

Referring to FIG. 23, a graph is shown illustrating drum position determination that may be performed using the data derived from switch 6004 in accordance with the present technology. In one aspect, the length of radial edge 6026 is multiplied by the t1/t2 ratio by controller 6010 for an individual target 6002 to obtain the distance measurement for the individual target 6002 at a specific angle of the target 6002 and drum 6050. It is to be appreciated that that the angle of the target 6002 is the same as the drum angle and represents the number of degrees the drum 6050 has rotated from (after detection of) the home flag position. Controller 6010 is configured to calculate the distance measurement for the targets 6002 at a plurality of angles (ranging from) 0-360° and fit the calculated distances at each angle to a sine function as shown in FIG. 23. It is to be appreciated that the distance measurements (the boxes in the chart) in FIG. 23 each correspond to a different target 6002 of the plurality of targets 6002. In this regard, each measurement corresponds to the target 6002 that is currently at the optical switch 6004 during the rotation. Fitting the calculated distances to the sine function reduces the noise in the individual distance readings. The amplitude of the sine fit indicates the offset of the drum position. For example, in FIG. 23, the amplitude is 0.9 mm, which represents the maximum offset of the drum position from its center of rotation. The phase of the sine fit indicates the angle of the drum relative to the home angle where the maximum offset occurs. For example, in the chart in FIG. 23, the phase is 45°, which represents the angle of the drum position. In this way, using the measurements from switch 6004 (and home sensor 6063), controller 6010 configured to determine the offset of the drum position and the angle of the drum position.

In use, the drum 6050 is operated in “test cycles”, which are associated with data collection that occurs during calibration of the drum 6050 and during normal use of the drum 6050. A test cycle is a process by which fluorescent data (from sensors 6062) is collected from the bottles in the drum 6050 and timing data is collected from the target sensor 6004. Each test cycle may include several rotations of the drum 6050, during which target ratios for each target 6002 and fluorescent data for the bottles in the drum 6050 are collected for each rotation of the drum 6050. Test cycles may be performed during calibration of the drum (e.g., in the manufacturing stage) or during normal use of the drum. Calibration may be performed during final checkout of the instrument in manufacturing with the drum 6050 filled with calibrator bottles. Data collected during a test cycle occurring during calibration is saved as drum normalization parameters (described below). Data is also collected during test cycles during normal use of the drum 6050 (e.g., to test culture bottles readings to detect positive bottles). Thus, test cycles are performed both during calibration and normal use of the drum 6050. The data collected during calibration and normal use is the same, but the data collected during calibration is saved as the drum normalization parameters. The test cycle process may be controlled by controller 6010, where a user may select whether a test cycle is being performed for calibration (and thus the data is stored as normalization parameters) or during normal use of the drum (and thus the data is stored for later adjustment, if necessary).

In one aspect, a column of drum 6050 includes reference bottles (also called calibrator bottles) fixed in all positions of the column of drum 6050. The properties and contents of these bottles are known. The reference bottles in the reference bottle column remain in place both during calibration and normal use of the drum 6050. In one aspect, the reference bottles in the reference bottle column are not removable by the user. During calibration, the remaining columns (other than the reference bottle column) may be filled with calibrator bottles. During normal use, the remaining columns (other than the reference bottle column) may be filled with culture bottles to be tested using measurement board 6060.

In one aspect, normalization parameters for the drum 6050 are collected during manufacturing in a test cycle and then used to normalize the fluorescent readings (obtained from sensors 6062) of media bottles stored in drum 6050 and the timing ratios calculated for each target 6002. For example, during calibration of the drum 6050 in manufacturing, all drum stations (receptacles to receive bottles) in the drum 6050 are loaded with calibrator bottles. As described above, a column of reference bottles is fixed in all positions of a reference bottle column in the drum 6050. In addition, all of the columns other than the reference bottle column of drum 6050 are filled with calibration bottles. Thus, all stations or receptacles in drum 6050 are filled with reference/calibrator bottles. During a test cycle, the drum 6050 is then turned at a constant measurement speed and fluorescent readings are taken via measurement board 6060 from the calibrators and reference bottles. Moreover, the timing ratios t1/t2 for each target 6002 are calculated using the timing data derived from switch 6004 for each of targets 6002 as they pass the measurement board 6060. The collection of fluorescent readings and timing ratios are stored in memory (e.g., memory 6012) as normalization parameters for the drum 6050.

Then, during each subsequent test cycle, e.g., during normal use of the drum 6050, the position of the drum 6050 is determined by controller 6010 and used to calculate the change in distance between each bottle in the drum and its corresponding sensor in measurement board 6060, relative to the same distance measured when the drum was normalized. Controller 6010 is configured to use the change in distance between a bottle and its corresponding sensor in measurement board 6060 to adjust the stored fluorescent readings from that bottle when a change in distance is measured to account for the change in distance. Adjustment is performed on the stored raw fluorescent readings for all of the bottles, including the reference bottles in the reference bottle column.

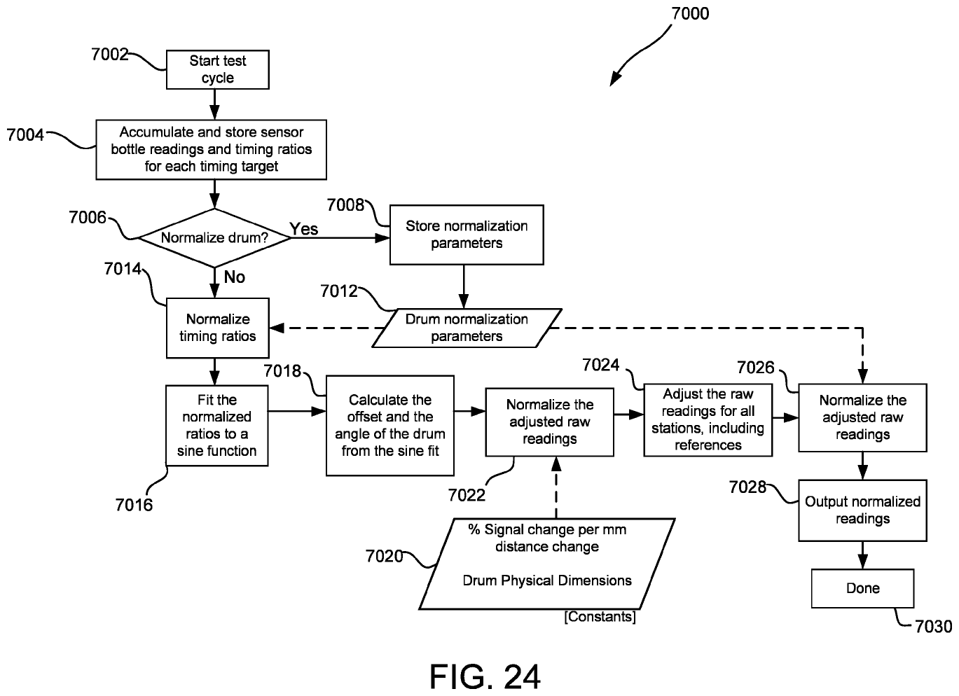

Referring to FIG. 24, a method 7000 for detecting the position or orientation of a rotating drum, such as drum 6050, relative to a stationary measurement board, such as measurement board 6060, and processing the stored measurements of the measurement board based on the detected position of the drum is shown in accordance with an aspect of the present technology. It is to be appreciated that the test cycles and steps of method 7000 may be performed during calibration of the drum 6050 and also during normal use of the drum 6050.

In step 7002, a test cycle for the blood culture apparatus is started. In one aspect, during the test cycle, all of the stations in the drum 6050 are loaded with calibrator bottles. A column of reference bottles is fixed in all positions of a reference bottle column in the drum 6050. The drum 6050 is turned in rotational direction 6070 at a predetermined and constant measurement speed.

In step 7004, during the test cycle, the timing ratios (t1/t2, described above) for each target 6002 are calculated by controller 6010 and accumulated and stored in memory (e.g., memory 6012 of system 6000 and/or another memory of the blood culture apparatus). Moreover, the readings of the sensors 6062 in the measurement board 6060 of the bottles stored in the receptacles of drum 6050 are also accumulated and stored in memory. As described above, the test cycle may include several rotations of the drum 6050.

In step 7006, it is determined whether to normalize the blood culture apparatus (i.e., the drum 6050) in relation to the measurement board 6060. Normalization of the drum 6050 is performed with calibrator bottles in all stations of the drum 6050. If it is determined to normalize the drum 6060 in step 7006, the method proceeds to step 7008. The determination at step 7006 may be based on controller 6010 receiving user input (e.g., from a technician during manufacturing) to normalize the drum 6050. In one aspect, normalization occurs in manufacturing when requested by the technician. The technician fills the drum 6050 with calibrator bottles and then requests the system (e.g., by input provided to controller 6010) to run a test cycle to collect calibration data from the drum 6050.

In step 7008, the target normalization ratios and drum station fluorescent normalization parameters are stored in memory, such as memory 6012 or a memory of measurement board 6060 or drum 6050. In one aspect, the target normalization ratios consist of 16 floating point numbers, each corresponding to a different target 6002. Together, the target normalization ratios and the drum station fluorescent normalization parameters form a set of normalization parameters. As described above, normalization parameters are determined from data collected when the drum 6050 is filled with calibrator bottles.

In step 7012, the drum station fluorescent normalization parameters are outputted for later use in steps 7014, 7026 when a test cycle is performed without normalizing the instrument and during normal use. Specifically, the target normalization ratios are provided for use in step 7014 and the drum station normalization parameters are provided for use in step 7026.

If it is determined not to normalize the drum (e.g., the drum is being operated during normal use) in relation to the measurement board 6060 in step 7006, the method proceeds to step 7014. In step 7014, the target ratios accumulated in step 7004 are normalized using the target normalization ratios. For example, in one aspect, the accumulated ratios for each target 6002 are averaged and the stored normalization ratios are subtracted from each averaged timing ratio. In this regard, the test cycle process includes several rotations of the drum, during which a target ratio for each target 6002 for each rotation is collected. The ratios for each target 6002 for all rotations are averaged to reduce noise in the ratio for each target 6002. Thus, an averaged target ratio for each target 6002 for the test cycle is calculated. Each resulting averaged target ratio is subtracted from its corresponding target normalization ratio (the normalization ratio corresponding to the specific target 6002). If, for example, the drum position has changed, this will produce a set of differences when each averaged target ratio is subtracted from its target normalization ratio. The set of differences is fit to a sine wave and indicates how much the drum has moved relative to its position when it was calibrated. The drum 6050 carrying calibrators from the test cycle is therefore considered the baseline position of the drum 6050, whether the drum 6050 is centered or not.

In step 7016, controller 6010 is configured to fit the normalized target timing ratios from step 7014 to a sine function as described above. This is performed once per test cycle. In step 7018, controller 6010 is configured to calculate the offset and angle of the drum 6050 from the sine fit of step 7016. As described above, the amplitude of the sine fit indicates the offset of the drum position and the phase of the sine fit indicates the angle of the drum position. The controller 6010 calculates the amplitude and phase of the sine fit. At this point in the method, the position of the timing targets 6002 (and the drum 6050) relative to where the timing target 6002 (and drum 6050) were when the drum 6050 was normalized is determined. It is to be appreciated that since the target timing ratios were normalized with the drum normalization parameters (in step 7014), the normalized target timing ratios represent the positions of each of the timing targets relative to their positions when the drum 6050 was normalized. Thus, the position of the timing targets and the position of the drum 6050 are the same.

In step 7022, controller 6010 is configured to calculate an adjustment value for each drum station. In one aspect, the adjustment value for each drum station is based on the individual station's angle from the drum offset angle, its height above the timing targets 6002, and the height of the pivot point 6051 at gearbox 6052 (see FIG. 17) relative to the timing targets 6002. As indicated at 7020, step 7022 receives as input several constants that are stored in memory. In one aspect, the constants comprise the percentage signal change of the readings from measurement board 6060 per mm distance change detected using the timing data from switch 6004 and the physical dimensions of the drum 6050, such as, the drum column angles and the drum row heights. It is to be appreciated that the percentage signal change of the readings from measurement board 6060 per mm distance change of the drum 6050 relative to the measurement board 6060 may be determined prior to performing method 7000 and stored in a memory. The physical dimensions of drum 6050 are known.

In step 7024, controller 6010 is configured to adjust the raw readings from all of the drum stations (receptacles) of drum 6050, including the readings from the reference bottles in the reference bottle column and the readings from non-reference bottles in the other columns (i.e., the bottles being tested). In one aspect, controller 6010 is configured to multiply the raw reading from each station by the adjustment value for that station obtained in 7022. The adjustment to the raw fluorescent readings from each location in the drum 6050 is selected such that changes in the adjusted readings are due only to changes in the bottle and not changes in position of the drum relative to the stationary measurement board 6060.

In step 7026, the adjusted raw readings from step 7024 are normalized using the drum station normalization parameters received from step 7012. In this regard, the difference between a fluorescent reading from a reference bottle in the reference bottle column during normalization and during normal use indicates a change in the measurement system (e.g., due to a change in position of the drum 6050). The change is applied by controller 6010 to the fluorescent readings for sample bottles in the same row (i.e., spanning a circle of stations around the drum) as the reference bottle to normalize the fluorescent reading for those sample bottles. This is performed for all the stations of the drum.

In step 7028, the normalized readings from step 7026 are outputted for processing and adjusting readings from measurement board 6060. After completion of step 7028, the method is finished in step 7030.

It is to be appreciated that the normalization process of FIG. 24 may be performed during manufacturing or during normal use of the blood culture apparatus and the results of FIG. 24 may be used for adjusting and correcting stored readings of measurement board 6060 in post-processing after it is detected that the drum position has changed during use of the drum.

It is to be appreciated that the present technology may be used to detect the position of the drum (and each receptacle and bottle contained therein) during any mode of operation of the drum (e.g., during calibration, normal operation, or any other mode).

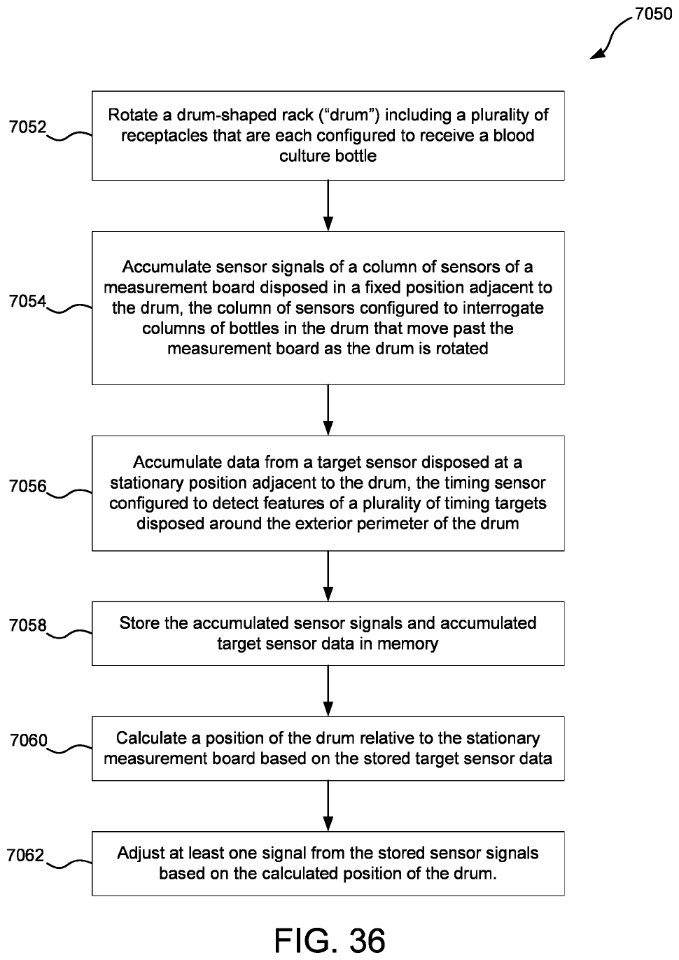

For example, FIG. 36 illustrates a method 7050 for detecting the position or orientation of a rotating drum, such as drum 6050, relative to a stationary measurement board, such as measurement board 6060, and processing the stored measurements of the measurement board based on the detected position of the drum in accordance with an aspect of the present technology.

In step 7052, a drum-shaped rack, e.g., drum 6050 having an exterior perimeter is rotated about an axis of rotation of the drum. The drum 6050 includes a plurality of receptacles configured to receive a blood culture bottle. Moreover, a plurality of timing targets 6002 are disposed around the exterior perimeter of the drum 6050 that rotate with drum 6050, as described above.

In step 7054, sensor signals from a column of sensors 6062 of a measurement board 6060 are accumulated. As described above, the measurement board 6060 is disposed in a fixed or stationary position adjacent or opposite to the exterior perimeter of the drum 6050. The column of sensor 6062 are configured to interrogate receptacles in columns in the drum 6050 column by column as each column moves past the measurement board 6060 as the drum 6050 is rotated about its axis of rotation.

In step 7056, data associated with a target sensor 6004 that is disposed at a fixed or stationary position adjacent to the drum 6050 is accumulated or obtained. As described above, the target sensor 6004 may be mounted to the measurement board 6060 and configured to detect geometric feature(s) (e.g., edges) of each of the timing targets 6002 as each timing target 6002 moves or rotates past (or through) the target sensor 6004 when the drum 6050 is rotated about the axis. The data associated with the target sensor 6004 may be data received directly from the target sensor 6004 or data derived from target sensor 6004. For example, the data may be the timing ratios described above.

In step 7058, the accumulated sensor signals from sensors 6062 and accumulated target sensor data from target sensor 6004 are stored in memory, such as memory 6012.

In step 7060, a controller or processor, such as controller 6010, is configured to calculate or determine a position (or orientation) of the drum 6050 relative to the stationary measurement board 6060 (or relative to another reference object that is stationary relative to drum 6050) based, at least in part, on the stored target sensor data from the target sensor 6004. As described above, the calculated position or orientation of the drum may comprise the drum offset (the distance of the center of the drum 6050 from the axis of rotation of the drum 6050) and the drum angle (the angle of the maximum drum offset relative to the home position of the targets 6002 and drum 6050). The drum offset and drum angle may be calculated by fitting the timing data (e.g., the timing ratios described above) to a sine function and determining the amplitude and phase of the sine fit. Step 7060 may further comprise determining, based on the position of the drum 6050, the position of each individual receptacle in the drum 6050 relative to a fixed or stationary reference object (e.g., the measurement board 6060). As described above, the physical dimensions of the drum 6050, including the positions/layout of each of the receptacles, are known. Moreover, the pivot point 6051 of the drum 6050 is known. From the position of the drum 6050 and the known dimensions and pivot point of the drum, the positions of each individual receptacle (or bottle therein) and their distance from the measurement board 6060 may be determined by controller 6010.

In step 7062, controller 6010 is configured to calculate an adjustment value and adjust at least one sensor signal of the sensor signals accumulated from sensors 6062 of measurement board 6060. It is to be appreciated that controller 6010 may be configured to determine if any adjustment is necessary prior to step 7062 by determining if there has been a change in position of the drum 6050 relative to the position of the drum 6050 during calibration of the drum 6050. If it is determined that an adjustment is necessary, controller 6010 is configured to calculate and adjustment value and adjust at least one (or all) of the sensor signals from sensors 6062 previously accumulated. As described above, the adjustment value may be based on the distance change (relative to a home position) between the measurement board 6060 and each bottle/receptacle of the drum 6050.

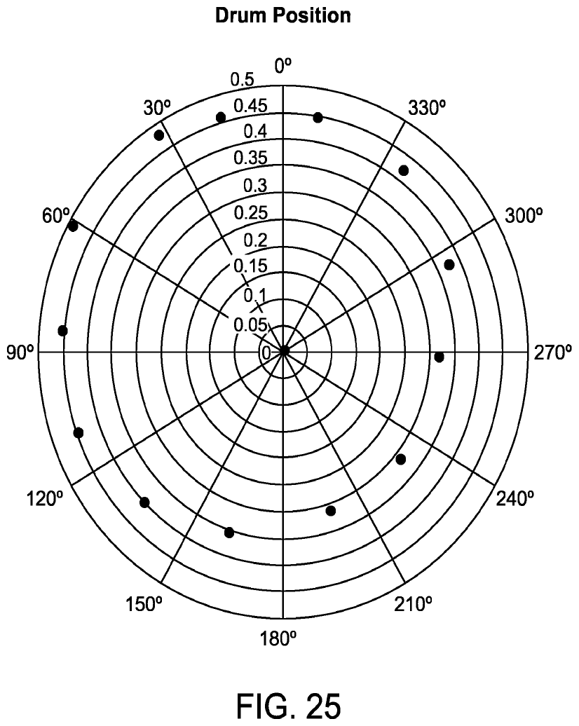

FIGS. 25-33 illustrates the results of several drum position detection tests performed using the techniques described above in relation to system 6000 and method 7000. Each of FIGS. 25-33 are discussed below.

FIG. 25 illustrates a radial plot of the position of drum 6050 under various loading conditions in accordance with the present technology. To obtain the points in the plot of FIG. 25, calibrator bottles were placed in receptacles around drum 6050. Weights were added to the drum 6050 centered on one column in the drum 6050. The weights were placed in different drum columns of the drum 6050 but kept in the same row and the effect on angular offset and distance offset was observed for each placement. Fluorescent readings (obtained from measurement board 6060) from the calibrator bottles, and drum position were collected for each weight placement, including for the scenario where no weights were placed. The plot in FIG. 25 shows the offsets and angles measured using switch 6004 and targets 6002 for each weight position. The center point represents no added weights. The offset measurements are in millimeters.

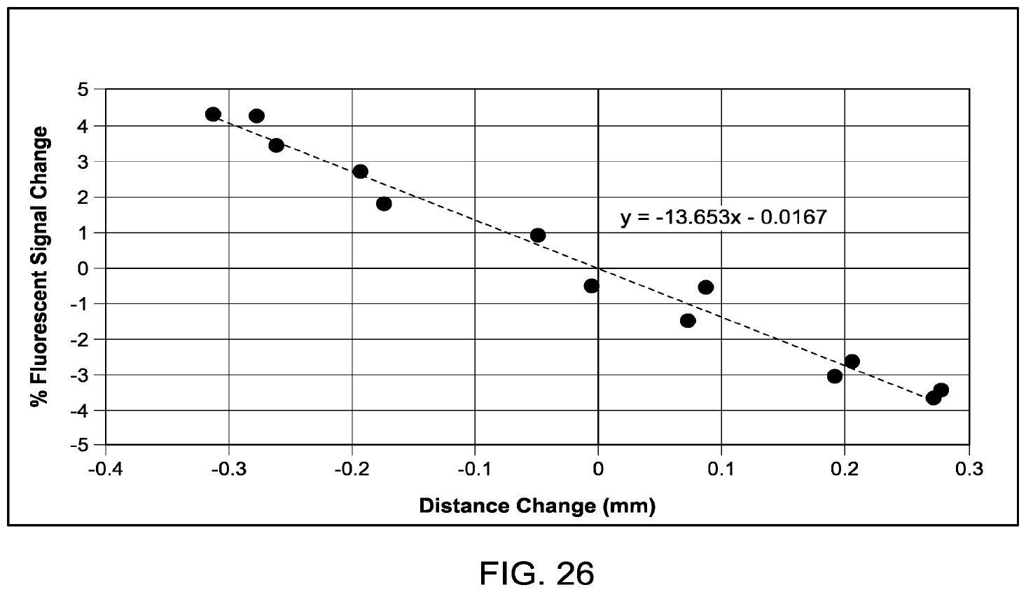

FIG. 26 is a graph in accordance with the present technology of the relationship between the percentage signal change of a measurement from board 6060 for a bottle stored in the drum 6050 and the change in distance between that bottle and the sensor on the board 6060 that reads the bottle. The slope of the line in the graph in FIG. 26 indicates the percent change in the fluorescent signal per millimeter change in bottle to sensor (on board 6060) distance. In the example of FIG. 26, the slope is-13.7%/mm.

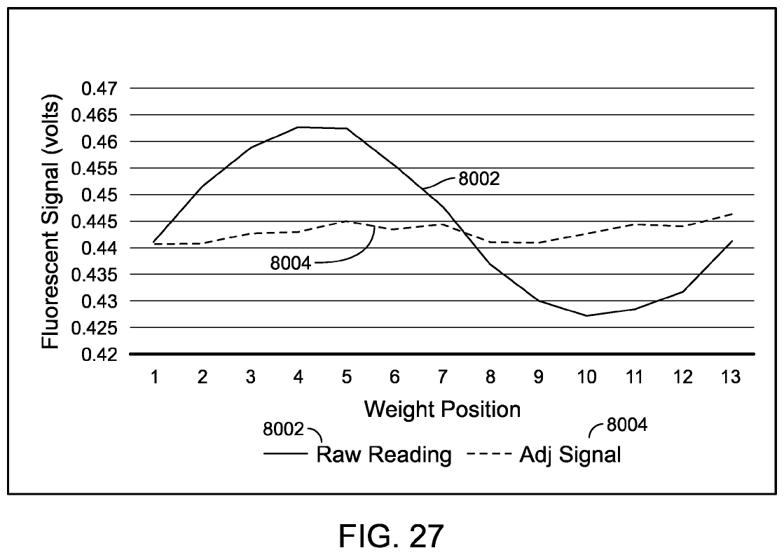

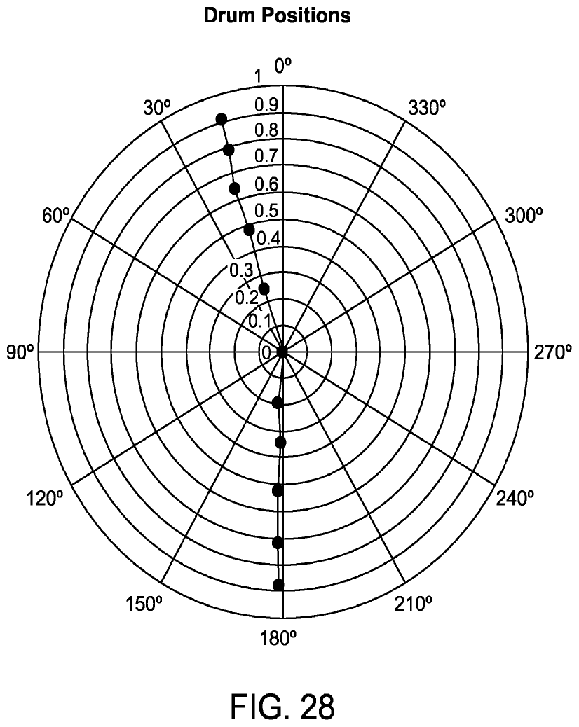

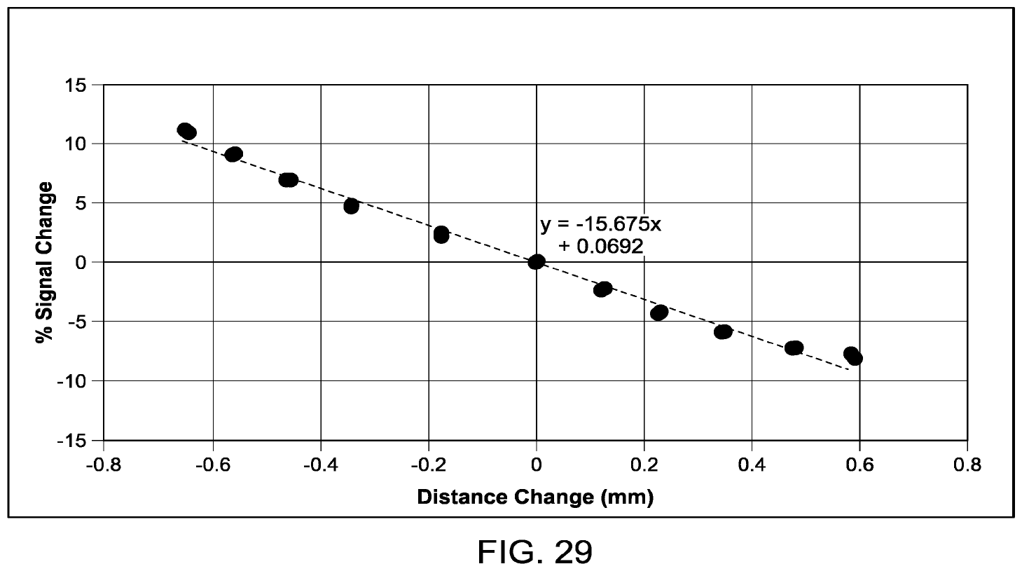

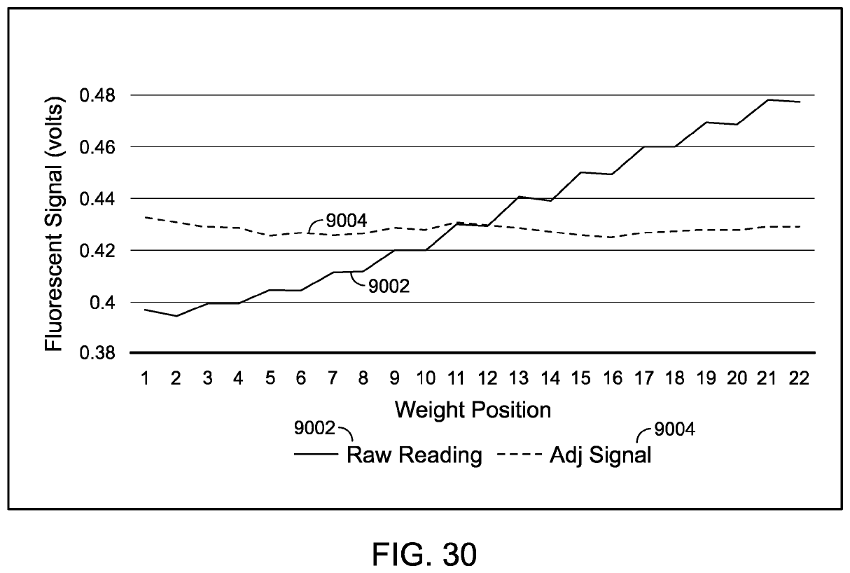

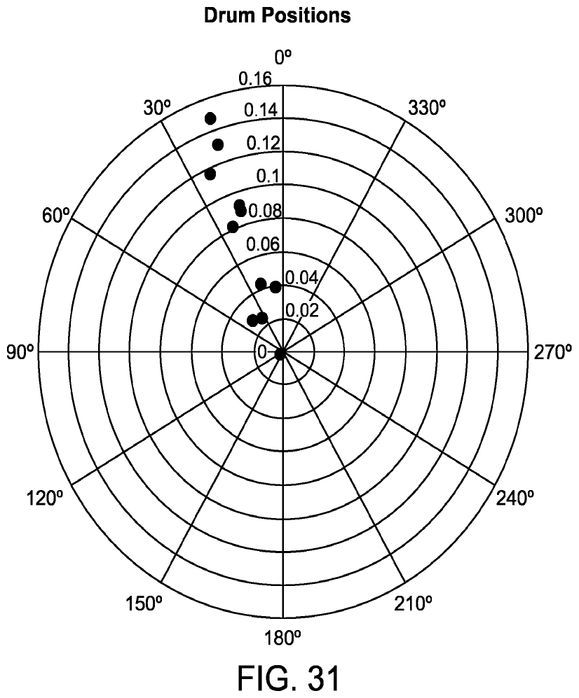

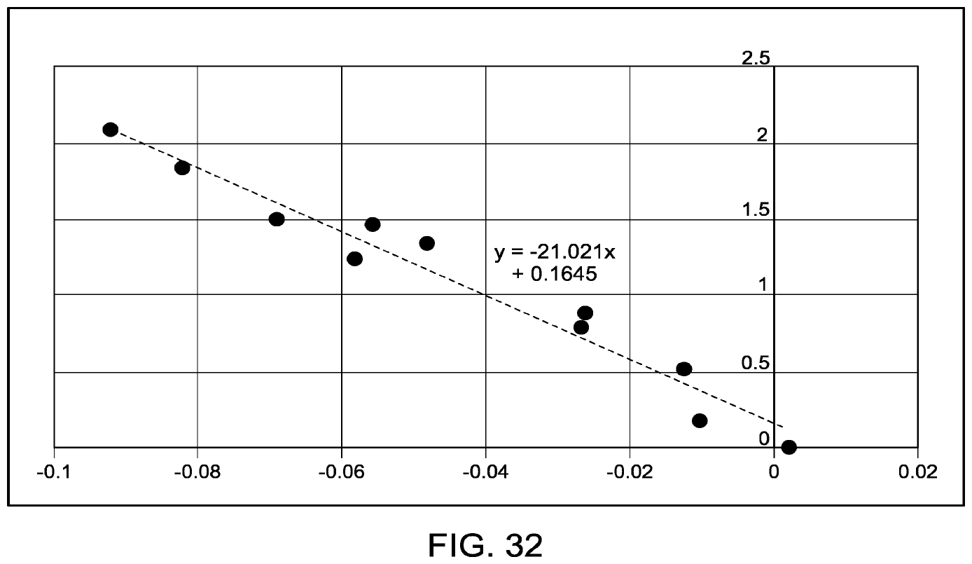

FIG. 27 is a graph in accordance with the present technology of a signal 8004 of a sensor on board 6060 that has been adjusted based on drum position changes detected using system 6000 and the techniques described above and a signal 8002 from the sensor on board 6060 (raw) unadjusted. The unadjusted signal 8002 was recorded as weights were moved to different positions around drum 6050. The adjusted signal 8004 was obtained based on the measured offset and angle of the drum 6050 using switch 6004 and target 6002 and the techniques described above and the bottle's position in the drum.

FIG. 28 illustrates a radial plot of the position of drum 6050 under various loading conditions in accordance with the present technology. Calibrator bottles were placed at random positions around the drum 6050. Steel shot filled bottles were placed symmetrically in relation to the rack column located at 0°. The weighted bottles were then progressively added to the opposite side of the drum 6050 symmetrically in relation to the rack column located at approximately 192°. The plot in FIG. 28 shows the offsets and the angles measured using the targets 6002 and switch 6004 for each weight configuration.

FIG. 29 is another graph in accordance with the present technology of the relationship between the percentage signal change of a measurement from board 6060 for a bottle stored in the drum 6050 and the change in distance between that bottle and the sensor on the board 6060 that reads the bottle. The slope of the line in the graph FIG. 29 indicates the percent change in the fluorescent signal per millimeter change in bottle to sensor (on board 6060) distance. In the example of FIG. 29, the slope is-15.7%/mm.

FIG. 30 is a graph in accordance with the present technology of a signal 9004 of a sensor on board 6060 that has been adjusted based on drum position changes detected using system 6000 and the techniques described above and a signal 9002 from the sensor on board 6060 (raw) unadjusted. The unadjusted signal 9002 was recorded as weights were moved to different positions around drum 6050. The adjusted signal 9004 was obtained based on the measured offset and angle of the drum 6050 using switch 6004 and target 6002 and the techniques described above and the bottle's position in the drum 6050.

FIG. 31 is a radial plot of the position of drum 6050 under various loading conditions in accordance with the present technology. Calibrator bottles were placed around the drum 6050 at random positions. Media bottles were added to one column of the drum 6050, e.g., column 10, and then placed individually in a range of rows, e.g., rows 1 through 5 of column 10. Then, media bottles were added to, e.g., columns 9 and 11 in pairs, in a range of rows, e.g., rows 1 through 5. The radial plot of FIG. 31 shows the offsets and angles measured using the targets 6002 and switch 6004 for each media bottle configuration.

FIG. 32 is another graph in accordance with the present technology of the relationship between the percentage signal change of a measurement from board 6060 for a bottle stored in the drum 6050 and the change in distance between that bottle and the sensor on the board 6060 that reads the bottle. The slope of the line in the graph in FIG. 31 illustrates the percent change in the fluorescent signal per millimeter change in bottle to sensor (on board 6060) distance. In the example of FIG. 29, the slope is −21.0%/mm. As with the previous graphs the x axis is the distance change in mm and the y axis is the % fluorescent signal change.

FIG. 33 is a graph in accordance with the present technology of a signal 10004 of a sensor on board 6060 that has been adjusted based on drum position changes detected using system 6000 and the techniques described above and a signal 10002 from the sensor on board 6060 (raw) unadjusted. The unadjusted signal 10002 was recorded as weight were moved to different positions around drum 6050. The adjusted signal 9004 was obtained based on the measured offset and angle of the drum 6050 using switch 6004 and target 6002 and the techniques described above and the bottle's position in the drum 6050.

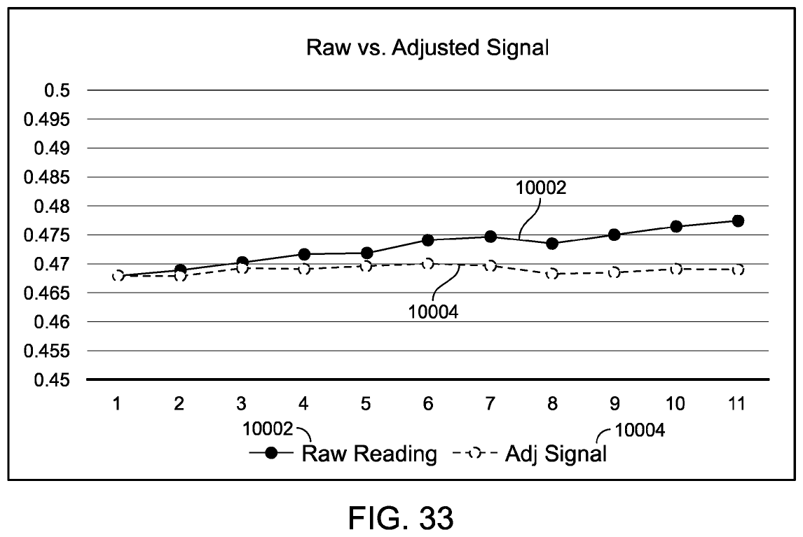

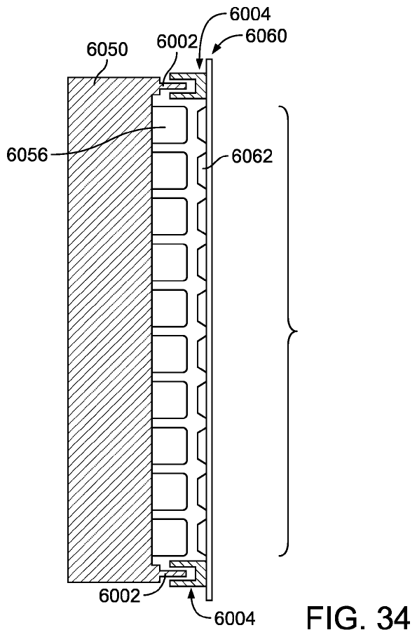

In one aspect, measurement system 6000 may include multiple sets of targets 6002 disposed at different locations on the exterior of drum 6050 and multiple sets of switches 6004 for detecting the timing relating to the passage of targets 6002 through the switches 6004 for detecting variations in distance and position in relation to the drum 6050. For example, referring to FIG. 34, drum 6050 is shown with a first plurality of targets 6002 disposed around the lower end of drum 6050 and a second plurality of targets 6002 disposed around the upper end of drum 6050. Switches 6004 are shown mounted to measurement board 6060 at corresponding locations that enable the first plurality of targets 6002 to pass through a first switch 6004 when drum 6050 is rotated and the second plurality of targets 6002 to pass through a second switch 6004 when the drum 6050 is rotated. The switches 6004 may be vertically aligned with a column of receptacles of the drum or a vertical section divider of the drum. The first plurality of targets 6002 and the second plurality of targets 6002 may also be vertically aligned, such that individual targets of each set align vertically. The targets 6002 and switches 6004 are configured in the manner described above. In this aspect, controller 6010 is configured to detect a change in the distance between any bottle 6056 in the column of bottles and its corresponding sensor in measurement board 6060 by interpolating the distances determined at the top and bottom of the measurement board and drum based on the layer of the drum the bottle is in. The detected distances may be used as described above for adjusting the signals of the sensors in the measurement board 6060 as needed.

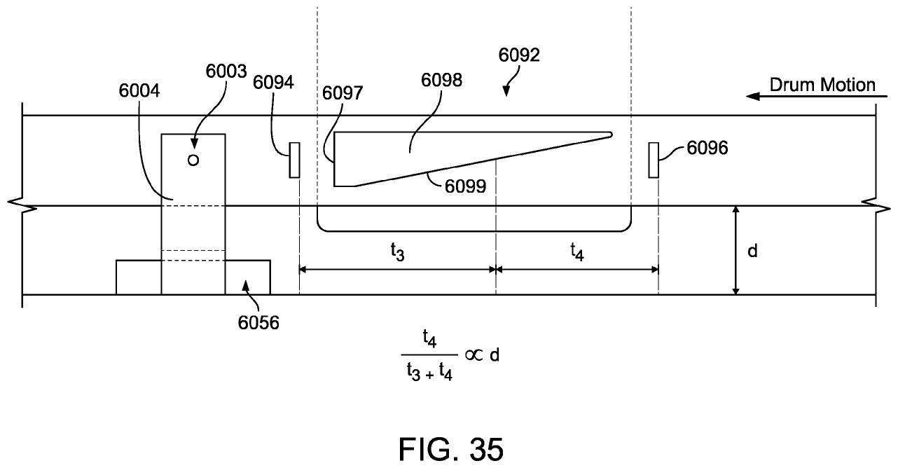

It is to be appreciated that, in other aspects, targets may be used with different geometry and different or additional physical features to targets 6002 shown in FIGS. 17-22 and described above. For example, referring to FIG. 35, a timing target 6092 for use in system 600 is shown in accordance with another aspect of the present technology. The timing target 6092 includes portions 6094, 6098, and 6096. The timing target 6092 is one of a plurality of timing targets 6092 that are disposed around (e.g., the lower end of) the drum 6050 such that they pass through switch 6004 during rotation of the drum. Portions 6094, 6096 extend radially from the drum and portion 6098 includes edges 6097, 6099, which are shaped in the same manner as edges 6024, 6026 of target 6002, described above. The portions 6094, 6098, and 6096 are configured to interrupt the light path of switch 6004 such that, in the manner described above, controller 6010 may detect the time elapsed between the state changes of switch 6004 as portions 6094, 6098, and 6096 interrupt the light path during rotations of the drum. From the state changes, controller 6010 may detect times t3 and t4 illustrated in FIG. 35. The following relationship holds true at any speed of the drum as long as the speed is constant:

$$\frac{{t}_{4}}{{t}_{3} + {t}_{4}} ∝ d \qquad ( Equation 1 )$$

As the distance between the drum and measurement board increases, t4 increases as t3+t4 remains constant, so the ratio in equation 1 increases. Both t3 and t4 are proportional to the drum speed, so the ratio is independent of the drum speed. Controller 6010 may use the ratio of equation 1 to detect the position of the drum and changes in distance between the drum and measurement and adjust the stored readings of the sensors of measurement board 6060 as described above.

It is to be appreciated that although the above-described drum position detection aspects of the present technology describe use of an optical switch 6004, in other aspects, other types of sensor(s) may be used with system 6000 for drum position detection. For example, the sensor(s) may be proximity detection device(s) that are configured to detect the proximity of the drum 6050 (or targets on the drum and the target features) to the measurement board 6060. The proximity detection device(s) may have a precision on the order of 0.1 mm over a range of 5-15 mm and are configured to accurately detect the distance in approximately (e.g., +/−25%) 1 millisecond as the target passes the proximity device.

In one aspect of the above-described drum detection techniques, the rotational speed of the drum is intended to be constant while the measurement board 6060 measures the bottles. However, in practice, such a constant measurement may not be guaranteed. In another aspect, as an alternative to measuring or determining the time between the interrupts from the optical switch 6004 is to measure the distance directly using an optical encoder strip. In this aspect, system 6000 includes an optical encoder strip that is mounted to the exterior of the drum 6050 and an encoder. In one aspect, the encoder is added to the measurement board 6060. In another aspect, the encoder is mounted to a stationary position exterior to the drum 6050 other than the measurement board 6060 such that the optical encoder strip passes the encoder. Controller 6010 or a controller or processor on the measurement board 6060 accumulates encoder counts as the drum 6050 rotates, and captures the number of encoder counts that occur in between the interrupts from the optical switch 6004. The ratio of the encoder counts replaces the timing to get the same ratio that is proportional to the distance between the drum 6050 and the measurement board 6060. Thus, in this aspect, controller 6010 or the controller/processor on the measurement board 6060 is configured to use the encoder counts and ratio of the encoder counts to determine changes to the drum position 6050 and changes in distance between the drum 6050 and the measurement board 6060.

It is to be appreciated that the above-described aspects relating to the drum position detection may also be used to detect potential shape changes or mechanical failures of the drum 6050. For example, as described above, the initial shape of the drum 6050, as assembled, is known. The initial shape may also be determined from the drum normalization parameters described above. Changes in position to the drum 6050 or changes in distance between the drum 6050 and the measurement board 6060 relative to the position or distance detected during manufacturing may be indicative of a change in shape to the drum 6050, such as, due to a loose mechanical connection of the drum 6050. Controller 6010 may detect these changes in shape based on detected changes in drum position or distance relative to the position or distance detected during manufacturing and may alert a user (e.g., via a communication signal sent from controller 6010 and/or communication module of the system) of the potential of a loose mechanical connection causing the change in shape of the drum 6050. In one aspect, a change to the initial shape may be assumed as a possible reason for a change in position of the drum or a change in distance between the drum and measurement board where the position/distances vary during two measurement each performed when the drum is completely unloaded. A change in shape may also be assumed where changes in position or distance are detected from drum models that do not typically wobble even when loaded differently or in an unbalanced manner.

While the drum position detection technology disclosed herein is described in relation to a blood culture apparatus including a rotating drum, the present technology may be implemented with other apparatuses or systems that include a rotating structure that rotates in relation to a stationary structure such that the change in position of the rotating structure relative to the stationary structure and changes in distance at various points between the rotating structure and the stationary structure may be detected and used for various purposes.

In one aspect, a system for detecting the position of a drum and the bottles carried in the drum is described. The system includes a drum-shaped rack, a measurement board, a plurality of timing targets, a target sensor, and a controller. The drum has an exterior perimeter and a plurality of receptacles that are each configured to receive a blood culture bottle. The exterior perimeter is disposed about an axis of rotation of the drum. The plurality of receptacles are arranged in the drum as an array of receptacles. The array has receptacles disposed both vertically and horizontally and the vertically aligned receptacles form a column and the horizontally aligned receptacles form a row. The measurement board is disposed at a stationary position adjacent to the drum and comprises a column of sensors configured to interrogate a column of bottles in the drum that moves past the measurement board as the drum is rotated about the axis. The plurality of timing targets are disposed around the exterior perimeter of the drum that rotate with the drum. The target sensor is disposed at a stationary position adjacent to the drum and is configured to detect one or more features of the timing targets as each timing target moves past the target sensor when the drum is rotated about the axis. The controller is configured to determine a position of the drum based on data from the target sensor.

In one aspect of the system, the determined position of the drum comprises a drum offset and a drum angle.

In one aspect of the system, the controller is configured to detect the position of the drum based on timing data associated with the detected features of the timing targets moving past the target sensor when the drum is rotated.

In one aspect of the system, the timing data comprises timing ratios associated with the plurality of timing targets.

In one aspect of the system, the controller is configured to normalize the timing ratios.

In one aspect of the system, the controller is configured to fit the normalized timing ratios to a sine function.

In one aspect of the system, the controller is configured to calculate the drum offset from an amplitude of the sine fit of the normalized timing ratios.

In one aspect of the system, the controller is configured to calculate the drum angle from a phase of the sine fit of the normalized timing ratios.

In one aspect of the system, the target sensor is an optical sensor.

In one aspect of the system, the optical sensor is configured to change states when a feature of the timing target interrupts a light path of the optical sensor.

In one aspect of the system, the controller is configured to detect the position of the drum based, at least in part, on state changes of the optical sensor.

In one aspect of the system, the one or more features of each timing target comprise a first edge and a second edge of the timing target.

In one aspect of the system, the first edge extends radially from the exterior perimeter of the drum and the second edge includes a first end and a second end, wherein a radial distance from the exterior perimeter of the drum to the second edge continuously decreases from the first end of the second edge to the second end of the second edge.

In one aspect of the system, the first end of the second edge connects to the first edge.

In one aspect of the system, the second end of the second edge connects to a first edge of an adjacent timing target of the plurality of timing targets.

In one aspect of the system, the plurality of timing targets are each aligned vertically with a column of receptacles of the drum.

In one aspect of the system, the controller is configured to adjust at least one signal of at least one sensor in the column of sensors in the measurement board based on the determined position of the drum.

In one aspect of the system, the at least one signal is a signal stored in a memory of the system.

In one aspect of the system, the drum has an upper end and a lower end and the plurality of timing targets are disposed proximately to the lower end of the drum.

In one aspect of the system, the target sensor is mounted to the measurement board.

In one aspect, a method for detecting the position of a drum and the bottles carried in the drum is described. The method includes: rotating a drum-shaped rack having an exterior perimeter about an axis of rotation of the drum-shaped rack, the drum-shaped rack having a plurality of receptacles arranged in an array of rows and columns, each receptacle configured to receive a blood culture bottle, and a sensor measurement board placed opposite an exterior perimeter of the drum-shaped rack, the sensor measurement board comprising a plurality of sensors arranged in a column such that each sensor in the sensor panel is aligned with a receptacle in the drum-shaped rack, wherein a plurality of timing targets are disposed around the exterior perimeter of the drum that rotate with the drum, each target comprising a geometric feature extending from the perimeter of the drum-shaped rack; accumulating sensor signals from the column of sensors of the measurement board that is disposed in a fixed position opposite the exterior perimeter of the drum-shaped rack, the column of sensors configured to interrogate receptacles in the drum-shaped rack column by column as each column moves past the measurement board as the drum-shaped rack is rotated about its axis; obtaining data from a target sensor disposed at a stationary position opposite to the exterior perimeter of the drum-shaped rack, the target sensor configured to detect the geometric feature of each timing target as each timing target rotates past the target sensor when the drum-shaped rack is rotated about the axis; storing the accumulated sensor signals and the target sensor data in memory; calculating a position of the drum relative to the stationary measurement board based on the stored target sensor data from the target sensor; and determining if at least one signal from the stored sensor signals requires adjustment based on the calculated position of the drum; and adjusting at least on signal from the stored sensor signals if it is determined that adjustment is required.

In one aspect of the method, the calculated position of the drum comprises a drum offset and a drum angle.

In one aspect of the method, the position of the drum is calculated based on timing data associated with the detected geometric feature of the timing targets moving past the target sensor when the drum is rotated.

In one aspect of the method, the timing data comprises timing ratios associated with the plurality of timing targets, wherein the timing ratios are based on the amount of time that a geometric feature activates the target sensor and the time between when a first geometric feature activates the target sensor and a following geometric feature activates the target sensor.

In one aspect of the method, the method further comprises normalizing the timing ratios.

In one aspect of the method, the method further comprises fitting the normalized timing ratios to a sine function.

In one aspect of the method, the method further comprises calculating the drum offset from an amplitude of the sine fit of the normalized timing ratios.

In one aspect of the method, the method further comprises calculating the drum angle from a phase of the sine fit of the normalized timing ratios.

In one aspect of the method, the target sensor is an optical sensor.

In one aspect of the method, the optical sensor is configured to change states when a geometric feature of the timing target interrupts a light path of the optical sensor.

In one aspect of the method, the position of the drum is calculated based, at least in part, on state changes of the optical sensor.

In one aspect of the method, the one or more geometric features of each timing target comprise a first edge and a second edge of the timing target.

In one aspect of the method, the first edge extends radially from the exterior perimeter of the drum and the second edge includes a first end and a second end, wherein a radial distance from the exterior perimeter of the drum to the second edge continuously decreases from the first end of the second edge to the second end of the second edge.

In one aspect of the method, the second end of the second edge connects to a first edge of an adjacent timing target of the plurality of timing targets.

In one aspect of the method, the plurality of timing targets are each aligned vertically with a column of receptacles of the drum.

In one aspect of the method, the drum has an upper end and a lower end and the plurality of timing targets are disposed proximately to the lower end of the drum.

In one aspect of the method, the target sensor is mounted to the measurement board.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive.

It will further be understood that any reference herein to subject matter known in the field does not, unless the contrary indication appears, constitute an admission that such subject matter is commonly known by those skilled in the art to which the present technology relates.

1. A system comprising:
a drum-shaped rack having an exterior perimeter, the drum having a plurality of receptacles, each receptacle configured to receive a blood culture bottle, wherein the exterior perimeter is disposed about an axis of rotation of the drum, wherein the plurality of receptacles are arranged in the drum as an array of receptacles, the array having receptacles disposed both vertically and horizontally, the vertical receptacles forming a column and the horizontal receptacles forming a row;
a measurement board that is disposed at a stationary position adjacent to the drum, the measurement board comprising a column of sensors configured to interrogate a column of bottles in the drum that moves past the measurement board as the drum is rotated about the axis;
a plurality of timing targets disposed around the exterior perimeter of the drum that rotate with the drum;
a target sensor disposed at a stationary position adjacent to the drum, the target sensor configured to detect one or more features of the timing targets as each timing target moves past the target sensor when the drum is rotated about the axis; and
a controller configured to determine a position of the drum based on data from the target sensor.

2. The system of claim 1, wherein the determined position of the drum comprises a drum offset and a drum angle.

3. The system of claim 2, wherein the controller is configured to detect the position of the drum based on timing data associated with the detected features of the timing targets moving past the target sensor when the drum is rotated.

4. The system of claim 3, wherein the timing data comprises timing ratios associated with the plurality of timing targets.

5. The system of claim 4, wherein the controller is configured to normalize the timing ratios.

6. The system of claim 5, wherein the controller is configured to fit the normalized timing ratios to a sine function.

7. The system of claim 6, wherein the controller is configured to calculate the drum offset from an amplitude of the sine fit of the normalized timing ratios.

8. The system of claim 6, wherein the controller is configured to calculate the drum angle from a phase of the sine fit of the normalized timing ratios.

9. The system of claim 1, wherein the target sensor is an optical sensor.

10. The system of claim 9, wherein the optical sensor is configured to change states when a feature of the timing target interrupts a light path of the optical sensor.

11. The system of claim 10, wherein the controller is configured to detect the position of the drum based, at least in part, on state changes of the optical sensor.

12. The system of claim 1, wherein the one or more features of each timing target comprise a first edge and a second edge of the timing target.

13. The system of claim 12, wherein the first edge extends radially from the exterior perimeter of the drum and the second edge includes a first end and a second end, wherein a radial distance from the exterior perimeter of the drum to the second edge continuously decreases from the first end of the second edge to the second end of the second edge.

14. The system of claim 13, wherein the first end of the second edge connects to the first edge.

15. The system of claim 14, wherein the second end of the second edge connects to a first edge of an adjacent timing target of the plurality of timing targets.

16. The system of claim 1, wherein the plurality of timing targets are each aligned vertically with a column of receptacles of the drum.

17. The system of claim 1, wherein the controller is configured to adjust at least one signal of at least one sensor in the column of sensors in the measurement board based on the determined position of the drum.

18. The system of claim 17, wherein the at least one signal is a signal stored in a memory of the system.

19. The system of claim 1, wherein the drum has an upper end and a lower end and the plurality of timing targets are disposed proximately to the lower end of the drum.

20. The system of claim 1, wherein the target sensor is mounted to the measurement board.

21. A method comprising:
rotating a drum-shaped rack having an exterior perimeter about an axis of rotation of the drum-shaped rack, the drum-shaped rack having a plurality of receptacles arranged in an array of rows and columns, each receptacle configured to receive a blood culture bottle, and a sensor measurement board placed opposite an exterior perimeter of the drum-shaped rack, the sensor measurement board comprising a plurality of sensors arranged in a column such that each sensor in the sensor measurement board is aligned with a receptacle in the drum-shaped rack, wherein a plurality of timing targets are disposed around the exterior perimeter of the drum that rotate with the drum, each target comprising a geometric feature extending from the perimeter of the drum-shaped rack;
accumulating sensor signals from the column of sensors of the measurement board that is disposed in a fixed position opposite the exterior perimeter of the drum-shaped rack, the column of sensors configured to interrogate receptacles in the drum-shaped rack, column by column, as each column moves past the measurement board as the drum-shaped rack is rotated about its axis;
obtaining data from a target sensor disposed at a stationary position opposite to the exterior perimeter of the drum-shaped rack, the target sensor configured to detect the geometric feature of each timing target as each timing target rotates past the target sensor when the drum-shaped rack is rotated about the axis;
storing the accumulated sensor signals and the target sensor data in memory;
calculating a position of the drum relative to the measurement board based on the stored target sensor data from the target sensor; and
determining if at least one signal from the stored sensor signals requires adjustment based on the calculated position of the drum; and
adjusting at least on signal from the stored sensor signals if it is determined that adjustment is required.

22. The method of claim 21, wherein the calculated position of the drum comprises a drum offset and a drum angle.

23. The method of claim 22, wherein the position of the drum is calculated based on timing data associated with the detected geometric feature of the timing targets moving past the target sensor when the drum is rotated.

24. The method of claim 23, wherein the timing data comprises timing ratios associated with the plurality of timing targets, wherein the timing ratios are based on an amount of time that a geometric feature activates the target sensor and the time between when a first geometric feature activates the target sensor and a following geometric feature activates the target sensor.

25. The method of claim 24, further comprising normalizing the timing ratios.

26. The method of claim 25, further comprising fitting the normalized timing ratios to a sine function.

27. The method of claim 26, further comprising calculating the drum offset from an amplitude of the sine fit of the normalized timing ratios.

28. The method of claim 26, further comprising calculating the drum angle from a phase of the sine fit of the normalized timing ratios.

29. The method of claim 21, wherein the target sensor is an optical sensor.

30. The method of claim 29, wherein the optical sensor is configured to change states when a geometric feature of the timing target interrupts a light path of the optical sensor.

31. The method of claim 30, wherein the position of the drum is calculated based, at least in part, on state changes of the optical sensor.

32. The method of claim 21, wherein the one or more geometric features of each timing target comprise a first edge and a second edge of the timing target.

33. The method of claim 32, wherein the first edge extends radially from the exterior perimeter of the drum and the second edge includes a first end and a second end, wherein a radial distance from the exterior perimeter of the drum to the second edge continuously decreases from the first end of the second edge to the second end of the second edge.

34. The method of claim 33, wherein the first end of the second edge connects to the first edge.

35. The method of claim 34, wherein the second end of the second edge connects to a first edge of an adjacent timing target of the plurality of timing targets.

36. The method of claim 21, wherein the plurality of timing targets are each aligned vertically with a column of receptacles of the drum.

37. The method of claim 21, wherein the drum has an upper end and a lower end and the plurality of timing targets are disposed proximately to the lower end of the drum.

38. The method of claim 21, wherein the target sensor is mounted to the measurement board.
